(19) United States
(12) Patent Application Publication
(10) Pub. No.: US 2026/0018243 A1
(43) Pub. Date: Jan. 15, 2026

(54) Technique For Training Artificial Intelligence Model By Using Interaction Data Between Protein And Ligand

(71) Applicant: SYNTEKABIO, INC., Daejeon (KR)

(72) Inventors: Jong Sun JUNG, Daejeon (KR); Hee Hoon JUNG, Seoul (KR); Ahn Jae CHUNG, Seoul (KR); Tae Seob LEE, Seoul (KR)

(73) Assignee: SYNTEKABIO, INC., Daejeon (KR)

(21) Appl. No.: 19/107,885

(22) PCT Filed: Apr. 4, 2024
(86) PCT No.: PCT/KR2024/004381

§ 371 Date: Aug. 22, 2025

(30) Foreign Application Priority Data
Apr. 5, 2023 (KR) 10-2023-0045014
Apr. 12, 2023 (KR) 10-2023-0048283
Jun. 27, 2023 (KR) 10-2023-0083064
Oct. 31, 2023 (KR) 10-2023-0148662
Apr. 2, 2024 (KR) 10-2024-0044448

(51) Int. Cl. G16B 15/30; G06N 20/00; G16B 40/20

(52) U.S. Cl. CPC G16B 15/30; G06N 20/00; G16B 40/20

(57) ABSTRACT

Disclosed is a method performed by a computing device. The method may include a method for training an artificial intelligence model by using interaction data between a protein and a ligand. The method may include: converting a binding structure between a ligand and a protein into at least one binding word in text form which is processable in an artificial intelligence-based Large Language Model (LLM); generating training data using the at least one binding word; and training the LLM using the training data.

### TECHNICAL FIELD

The present disclosure relates to artificial intelligence technology, and more particularly, to training an artificial intelligence model using interaction data between a protein and a ligand.

### BACKGROUND ART

An interaction between a receptor protein and a ligand is key to mediating various functions in living organisms. As diverse as the functions of living organisms are, there are a wide variety of acceptor proteins and ligands that target them across a wide range of species and functional systems. Identifying and designing ligands that can bind to target acceptor proteins that mediate various functions is one of the major tasks of modern pharmacology. Therefore, interest in life sciences is focused on predicting or broadening the view of the interaction between acceptor proteins and ligands, especially chemical bonds.

The development of artificial intelligence technology has also brought about a new paradigm in life science research methodology. Various artificial intelligence models, such as the convolutional neural network (CNN) specialized in image processing and the large language model (LLM), a natural language processing model, are being incorporated into life science research methodologies.

For example, the Large Language Model (LLM) refers to a natural language processing model that is pre-trained using large text data. A generative pre-trained transformer (GPT), which belongs to the large language model, is a natural language processing artificial intelligence model developed by OpenAI, and shows good performance in various natural language processing tasks through pre-training using a large amount of text data.

Further, DrugGPT presented by Yuesen Li et al., enables inference using natural language processing models to design ligands based on specific protein sequences. Here, DrugGPT uses ligands denoted by SMILES independently from the protein amino acid sequences in the database as pre-training data. The Simplified Molecular Input Line Entry System (SMILES) is one of the notations for representing chemical substances. The SMILES character strings can represent molecules using the element symbols of their atoms. For example, a water molecule, which is made up of oxygen and hydrogen atoms, can be represented using SMILES notation as O—H—H. However, the SMILES has a limitation in compressing accurate three-dimensional spatial structures. The protein-ligand interaction is a complex and delicate process that occurs in three-dimensional space, and predicting the protein-ligand interaction requires preserving information beyond atomic properties, chemical bonds, and electrochemical charges.

In this regard, Korean Patent Registration No. 10-2617957 is contrived.

### DISCLOSURE

### Technical Problem

The present disclosure is contrived in response to the above-described background art, and has been made in an effort to train an artificial intelligence model by converting a binding structure between a protein and a ligand to a form to minimize a loss of complex interaction data in three-dimensional space.

Technical objects of the present disclosure are not restricted to the technical object mentioned above. Other unmentioned technical objects will be apparently appreciated by those skilled in the art by referencing the following description.

### Technical Solution

According to an embodiment of the present disclosure, disclosed is a method performed by a computing device. The method may include: converting, a binding structure between a ligand and a protein into at least one binding word in text form which is processable in an artificial intelligence-based Large Language Model (LLM); generating, training data using the at least one binding word; and training, the Large Language Model (LLM) using the training data.

In an embodiment, the converting may include converting the binding structure between a binding part of the ligand and a residue of the protein into the at least one binding word.

In an embodiment, the converting may include converting the binding structure, which represents an interaction of an electron donor and an electron acceptor between the binding part of the ligand and the residue of the protein into, the at least one binding word.

In an embodiment, the binding word may include a first sub-binding word representing an interaction from a perspective of the ligand on the binding structure; and a second sub-binding word representing an interaction from a perspective of the protein on the binding structure.

In an embodiment, the converting may include converting the binding structure between the ligand and the protein into the at least one binding word by concatenating the first sub-binding word and the second sub-binding word.

In an embodiment, the first sub-binding word may include a first part representing whether a role of a binding atom of the ligand on the binding structure is an electron donor or an electron acceptor; a second part identifying the binding atom of the ligand on the binding structure; and a third part identifying at least one proximal atom located proximal to the binding atom of the ligand on the binding structure.

In an embodiment, the first sub-binding word is a concatenated form of a first information representing whether a role of a binding atom of the ligand on the binding structure is an electron donor or an electron acceptor; a second information identifying a binding form formed by the binding atom of the ligand with other atoms of the ligand on the binding structure; a third information identifying a first proximal atom located proximal to the binding atom of the ligand and a binding form formed by the first proximal atom with other atoms of the ligand on the binding structure; a fourth information identifying the binding atom of the ligand and the binding form formed by the binding atom with other atoms of the ligand on the binding structure; and a fifth information identifying a second proximal atom located proximal to the binding atom of the ligand and a binding form formed by the second proximal atom with other atoms of the ligand on the binding structure.

In an embodiment, the second sub-binding word may include a first part identifying a binding amino acid of the protein on the binding structure; a second part identifying a receptor binding atom obtained from the binding amino acid; and a third part identifying at least one receptor proximal atom located proximal to the receptor binding atom.

In an embodiment, the at least one receptor proximal atom may include a first receptor proximal atom and a second receptor proximal atom, and in the third part identifying the at least one receptor proximal atom located proximal to the receptor binding atom, the first receptor proximal atom, the receptor binding atom, and the second receptor proximal atom may concatenated in the order of the first receptor proximal atom, the receptor binding atom, and the second receptor proximal atom.

In an embodiment, the first sub-binding word and the second sub-binding word, which constitute the binding word, are concatenated through a first expression to represent the binding word; the parts or information which constitute the first sub-binding word are concatenated through a second expression to represent the first sub-binding word; the parts or information which constitute the second sub-binding word are concatenated through the second expression to represent the second sub-binding word; and the first expression and the second expression are different from each other.

In an embodiment, the binding structure between the ligand and the protein may be a three-dimensional binding structure, and the at least one binding word may be one-dimensional data.

In an embodiment, the generating the training data may include generating a binding sentence representing a binding structure between a single protein residue and all ligand fragments binding to the single protein residue by combining a plurality of binding words; and generating the training data including the binding sentence.

In an embodiment, the generating the training data may include generating a binding paragraph representing a binding structure between a binding pocket of a single protein and each of all ligand fragments bound to the binding pocket by combining a plurality of binding sentences; and generating the training data including the binding paragraph.

In an embodiment, the generating the training data including the binding sentence may include generating the training data by annotating, to the binding sentences, identification information of protein, species information, and identification information of ligand corresponding to each of the binding sentences.

In an embodiment, the training the LLM using the training data may include tokenizing the training data. In addition, the tokenizing may include a word-based tokenization, in which each of the binding words acts as a token to form a vocabulary; and a byte-pair encoding tokenization, in which all words included in each binding paragraph are connected by a space character, and each binding paragraph is separated by a newline character.

The method disclosed in an embodiment may further include: extracting a plurality of first embedding vectors from at least one layer of the trained LLM; reducing dimensionality of each of the first embedding vectors to obtain a plurality of second embedding vectors; clustering the second embedding vectors or calculating a distance of each of the second embedding vectors in vector space; and determining a similarity of characteristics of proteins, a similarity of characteristics of ligands, and a binding potential between a protein and a ligand based on a result of the clustering or the distance. Here, the characteristics may include structural characteristics, functional characteristics, and genetic characteristics.

In an embodiment, the method may further include determining a single protein as a multi-functional protein when a plurality of embedding vectors corresponding to the single protein exist in a plurality of clusters as a result of the clustering.

In an embodiment, the method may further include determining additional uses of drugs corresponding to the ligands using the similarity of the characteristics of ligands.

In an embodiment, a computer program stored in a computer readable storage medium is disclosed. The computer program causes a computing device to perform following operations when executed by the computing device, and the operation may include: converting, a binding structure between a ligand and a protein into at least one binding word in text form which is processable in LLM; generating, training data using the at least one binding word; and training, the LLM using the training data.

A computing device according to an embodiment is disclosed. The computing device may include: at least one processor; and a memory. The at least one processor may perform: converting, a binding structure between a ligand and a protein into at least one binding word in text form which is processable in a LLM; generating training data using the at least one binding word; and training, the LLM using the training data.

### Advantageous Effects

According to an embodiment of the present disclosure, a method and an apparatus can train an artificial intelligence model by converting a binding structure between a protein and a ligand to a form to minimize a loss of complex interaction data in three-dimensional space.

### DESCRIPTION OF DRAWINGS

FIG. 1 schematically illustrates a block diagram of a computing device according to an embodiment of the present disclosure.

FIG. 2 illustrates an exemplary structure of an artificial intelligence-based model according to an embodiment of the present disclosure.

FIG. 3 exemplarily illustrates a method for training a large language model which processes a binding structure of a ligand and a protein according to an embodiment of the present disclosure.

FIG. 4 exemplarily illustrates a method using an embedding vector obtained from the trained large language model according to an embodiment of the present disclosure.

FIG. 5 illustrates a binding structure including a binding part in the ligand and a protein residue that is target of a conversion according to an embodiment of the present disclosure.

FIG. 6 exemplarily illustrates a binding form in which a binding atom or a proximal atom of the ligand, or a receptor binding atom or proximal atom forms with other atoms within the ligand or the receptor according to an embodiment of the present disclosure.

FIG. 7 exemplarily illustrates a conversion of the binding structure of the ligand and the protein to a text format processable in the large language model according to an embodiment of the present disclosure.

FIG. 8 additionally illustrates a conversion of the binding structure of the ligand and the protein to a text form processable in the large language model according to an embodiment of the present disclosure.

FIG. 9 illustrates an example of a binding word, a binding sentence, and a binding paragraph which is a converted version of the binding structure of the ligand and the protein to the text form processable in the large language model according to an embodiment of the present disclosure.

FIG. 10 illustrates an example of bindings constituting binding sentences according to an embodiment of the present disclosure.

FIG. 11 exemplarily illustrates a clustering result of an embedding vector extracted from a trained large language model according to an embodiment of the present disclosure.

FIG. 12 exemplarily illustrates a hierarchical clustering result and results sorted for each biological function according to an embodiment of the present disclosure.

FIG. 13 exemplarily illustrates a result in which proteins classified as multifunctional proteins are scattered in multiple clusters according to an embodiment of the present disclosure.

FIG. 14 illustrates an example in which shared bindings with other proteins are output when a query is input into a large language model trained by an embodiment of the present disclosure.

FIG. 15 exemplarily illustrates a result of determining a ligand that may be used as a common substrate from the results returned by the large language model trained by an embodiment of the present disclosure.

FIG. 16 is a schematic view of a computing environment according to an embodiment of the present disclosure.

### MODE FOR INVENTION

Various embodiments will be now disclosed with reference to drawings. In this disclosure, multiple detailed matters will be disclosed in order to help comprehensive appreciation of one or more aspects. In describing the present invention, it should be noted that configurations not directly related to the technical gist of the present disclosure are omitted within the scope of not dispersing the technical gist of the present disclosure. In addition, terms or words used in this specification and claims should be interpreted as meanings and concepts consistent with the technical idea of the present disclosure based on the principle that the inventor can define the concept of appropriate terms to explain his or her invention in the best way.

“Component”, “module”, “system”, “unit”, and the like which are terms used in the specification refer to a computer-related entity, hardware, firmware, software, and a combination of the software and the hardware, or execution of the software. For example, the component may be a processing process executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

A term “or” intends to mean comprehensive “or”, not exclusive “or”. That is, unless otherwise specified or when it is unclear in context, “X uses A or B” intends to mean one of the natural comprehensive substitutions. That is, when X uses A, X uses B, or X uses both A and B, “X uses A or B” may be applied to any one among the cases. Further, a term “and/or” used in the present specification shall be understood to designate and include all of the possible combinations of one or more items among the listed relevant items.

A term “include”, “comprise” and/or “including”, “comprising” shall be understood as meaning that a corresponding characteristic and/or a constituent element exists. Further, a term “include”, “comprise” and/or “including”, “comprising” means that a corresponding characteristic and/or a constituent element exists, but it shall be understood that the existence or an addition of one or more other characteristics, constituent elements, and/or a group thereof is not excluded. Further, unless otherwise specified or when it is unclear that a single form is indicated in context, the singular shall be construed to generally mean “one or more” in the present specification and the claims.

The term “at least one of A or B” or “at least one of A and B” should be interpreted to mean “a case wherein only A is included”, “a case where only B is included”, or “a case where A and B are combined”.

Those skilled in the art need to recognize that various illustrative logical blocks, configurations, modules, circuits, means, logic, and algorithm steps described in connection with the exemplary embodiments disclosed herein may be additionally implemented as electronic hardware, computer software, or combinations of both sides. To clearly illustrate the interchangeability of hardware and software, various illustrative components, blocks, configurations, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each specific application. However, such implementation decisions should not be interpreted as causing a departure from the scope of the present disclosure.

The description of the presented exemplary embodiments has been provided to allow one skilled in the art to use or implement the present invention. Various modifications to the exemplary embodiments will be apparent to one skilled in the art. The general principles defined herein may be applied to other exemplary embodiments without departing from the scope of the present disclosure. Therefore, the present invention is not limited to the exemplary embodiments presented herein. The present invention should be interpreted within the broadest scope consistent with the principles and novel features presented herein.

In the present disclosure, terms represented by N-th such as first, second, or third are used for distinguishing at least one entity. For example, entities expressed as first and second may be the same as each other or different from each other.

There are many different types of proteins in order to perform various functions within living organisms. Among them, receptor proteins are proteins that exist on a cell surface or inside the cell and detect external signals and transmit those signals internally to control a cell function. For example, chemical substances or hormones generated outside the cell are bound to receptors and are activated, which in turn activates various signal transmission paths inside the cell, thereby regulating the cell's response. Activation of receptors often activates the signal transmission paths. The signal transmission paths which are a series of biochemical reaction processes serve to receive a specific signal inside the cell and promote a response to the corresponding signal.

A ligand is a chemical substance that acts as a substrate for a receptor protein. The receptor protein is activated in response to chemical substances or signals generated from the external environment, and the activation is mediated by binding to a ligand that acts specifically on a receptor. Ligands, such as hormones, microbial toxins, and drugs, bound to receptor proteins to form ligand-receptor complexes, and this binding activates or inhibits signal transmission paths within cells, inducing chemical, physiological, or biological changes.

Ligand binding is usually very specific, binding only to a binding pocket which is a specific site of the receptor protein. This specificity is important for the receptor protein to identify a specific signal, and regulate a state of a cell in response thereto.

The binding pocket of the receptor protein is mainly exposed on the surface of the protein, and there is a specific three-dimensional arrangement formed by protein residues. In general, 20 types of protein residues are found in organisms, and combinations of the residues allow the receptor proteins to interact specifically with different types of ligands.

Interactions that occur between proteins and ligands include electron donor-acceptor interactions. An electron donor is a molecule that transfers electrons, and an electron acceptor is a molecule that accepts electrons. The interaction between these two molecules occurs through the transfer of electrons. For example, a protein residue may act as the electron donor, and a chemical structure of the ligand may act as the electron acceptor that may accept the electrons. Conversely, a reaction may also occur in which the protein residue acts as the electron acceptor and the ligand acts as the electron donor. The electron donor-electron acceptor interaction between the protein and the ligand may form a strong intermolecular binding, and the interaction is required for regulating the binding between the proteins and the ligand, regulating a physiological signaling pathway, and regulating a function of a living organism.

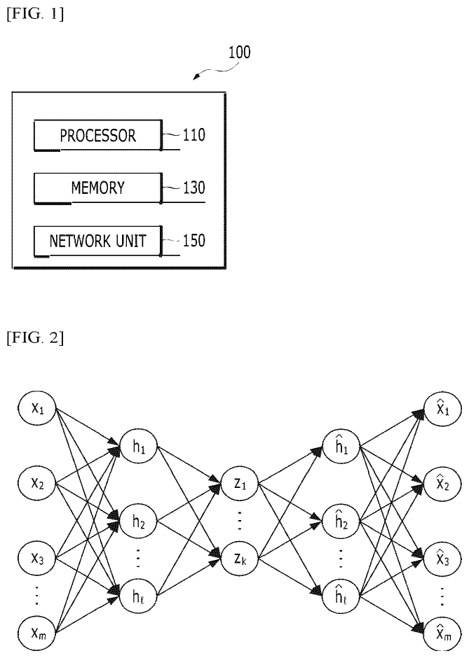

FIG. 1 schematically illustrates a block diagram of a computing device 100 according to an embodiment of the present disclosure.

The computing device 100 according to an embodiment of the present disclosure may include a processor 110 and a memory 130.

A configuration of the computing device 100 illustrated in FIG. 1 is only an example shown through simplification. In an embodiment of the present disclosure, the computing device 100 may include other components for performing the computing environment of the computing device 100, and only some of the disclosed components may constitute the computing device 100.

In the present disclosure, the computing device 100 may mean any type of node constituting a system for implementing embodiments of the present disclosure. The computing device 100 may mean any type of user terminal or any type of server. The components of the computing device 100 may be exemplary and some components may be excluded, or an additional component may be included in the computing device 100. As an example, when the computing device 100 includes a user terminal, an output unit (not illustrated) and an input unit (not illustrated) may be included in a scope of the computing device 100.

The computing device 100 in the present disclosure may perform technical features according to embodiments of the present disclosure to be described below. For example, the computing device 100 may train an artificial intelligence-based large language model using input data corresponding to a binding structure between a ligand and a protein. For example, the computing device 100 may generate a prediction result including a separate binding structure corresponding to the input data by using an artificial intelligence-based large language model using input data corresponding to a binding structure between a ligand and a protein. For example, the computing device 100 may generate a prediction result that includes separate binding structures functionally corresponding to a specific binding structure between the ligand and the protein.

According to an embodiment of the present disclosure, the processor 110 may also perform an operation for learning a neural network. The processor 110 may perform calculations for learning the neural network, which include processing of input data for learning in deep learning (DL), extracting a feature from the input data, calculating an error, updating a weight of the neural network using backpropagation, and the like. At least one of the CPU, GPGPU, and TPU of the processor 110 may process learning of a network function. For example, both the CPU and the GPGPU may process the learning of the network function and data classification using the network function. Further, in an embodiment of the present disclosure, processors of the plurality of computing devices may be used together to process the learning of the network function and the data classification using the network function. Further, the computer program executed in the computing device according to an embodiment of the present disclosure may be a CPU, GPGPU, or TPU executable program.

Additionally, the processor 110 may generally process an overall operation of the computing device 100. For example, the processor 110 processes data, information, signals, and the like input or output through the components included in the computing device 100 or drives the application program stored in a storage unit to provide or process information or a function appropriate for the user.

According to an embodiment of the present disclosure, the memory 130 may store any type of information generated or determined by the processor 110 or any type of information received by the computing device 100. According to an embodiment of the present disclosure, the memory 130 may be a storage medium that stores computer software which allows the processor 110 to perform the operations according to the embodiments of the present disclosure. Therefore, the memory 130 may mean computer-readable media for storing software codes required for performing the embodiments of the present disclosure, data which become execution targets of the codes, and execution results of the codes.

According to an embodiment of the present disclosure, the memory 130 may mean any type of storage medium, and include, for example, at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the memory 130 used in the present disclosure is not limited to the examples.

In the present disclosure, the communication unit (not illustrated) may be configured regardless of communication modes such as wired and wireless modes and constituted by various communication networks including a personal area network (PAN), a wide area network (WAN), and the like. Further, the network unit 150 may operate based on known World Wide Web (WWW) and may adopt a wireless transmission technology used for short-distance communication, such as infrared data association (IrDA) or Bluetooth.

The computing device 100 in the present disclosure may include any type of user terminal and/or any type of server. Therefore, the embodiments of the present disclosure may be performed by the server and/or the user terminal.

The user terminal may include any type of terminal which is capable of interacting with the server or another computing device. The user terminal may include, for example, a mobile phone, a smart phone, a laptop computer, personal digital assistants (PDA), a slate PC, a tablet PC, and an Ultrabook.

The server may include, for example, any type of computing system or computing device such as a microprocessor, a mainframe computer, a digital processor, a portable device, and a device controller.

In an additional embodiment, the server may also mean an entity that stores and manages protein information, ligand information, peptide sequence information, base sequence information, or genetic information. The server may include a storage unit (not illustrated) for storing immunopeptidome information, peptide sequence information, position-specific amino acid identifiers information, base sequence information, genetic information, or reliability information of a database (e.g., PDB, UniProt, Pocketome, VDJdb, IMGT), and the storage unit may be included in the server or may exist under the management of the server. As another example, the storage unit may also be present outside the server, and implemented in a form which is capable of communicating with the server. In this case, the storage unit may be managed and controlled by another external server different from the server.

FIG. 2 illustrates an exemplary structure of an artificial intelligence-based model according to an embodiment of the present disclosure.

Throughout this specification, a language model, a large language model, an artificial intelligence-based large language model, an artificial intelligence model, an artificial intelligence-based model, a computation model, a neural network, a network function, and a neural network may be used as the same meaning.

The neural network may be generally constituted by an aggregate of calculation units which are mutually connected to each other, which may be called nodes. The nodes may also be called neurons. The neural network is configured to include one or more nodes. The nodes (alternatively, neurons) constituting the neural networks may be connected to each other by one or more links.

In the neural network, one or more nodes connected through the link may relatively form the relationship of an input node and an output node. Concepts of the input node and the output node are relative and a predetermined node which has the output node relationship with respect to one node may have the input node relationship with another node and vice versa. As described above, the relationship of the input node to the output node may be generated based on the link. One or more output nodes may be connected to one input node through the link and vice versa.

In the relationship of the input node and the output node connected through one link, a value of data of the output node may be determined based on data input in the input node. Here, a link connecting the input node and the output node to each other may have a weight. The weight may be variable and the weight is variable by a user or an algorithm in order for the neural network to perform a desired function. For example, when one or more input nodes are mutually connected to one output node by the respective links, the output node may determine an output node value based on values input in the input nodes connected with the output node and the weights set in the links corresponding to the respective input nodes.

As described above, in the neural network, one or more nodes are connected to each other through one or more links to form a relationship of the input node and output node in the neural network. A characteristic of the neural network may be determined according to the number of nodes, the number of links, correlations between the nodes and the links, and values of the weights granted to the respective links in the neural network. For example, when the same number of nodes and links exist and there are two neural networks in which the weight values of the links are different from each other, it may be recognized that two neural networks are different from each other.

The neural network may be constituted by a set of one or more nodes. A subset of the nodes constituting the neural network may constitute a layer. Some of the nodes constituting the neural network may constitute one layer based on the distances from the initial input node. For example, a set of nodes of which distance from the initial input node is n may constitute n layers. The distance from the initial input node may be defined by the minimum number of links which should be passed through for reaching the corresponding node from the initial input node. However, definition of the layer is predetermined for description and the order of the layer in the neural network may be defined by a method different from the aforementioned method. For example, the layers of the nodes may be defined by the distance from a final output node.

In an embodiment of the present disclosure, a set of neurons or nodes may be defined as an expression layer.

The initial input node may mean one or more nodes in which data is directly input without passing through the links in the relationships with other nodes among the nodes in the neural network. Alternatively, in the neural network, in the relationship between the nodes based on the link, the initial input node may mean nodes which do not have other input nodes connected through the links. Similarly thereto, the final output node may mean one or more nodes which do not have the output node in the relationship with other nodes among the nodes in the neural network. Further, a hidden node may mean nodes constituting the neural network other than the initial input node and the final output node.

In the neural network according to an embodiment of the present disclosure, the number of nodes of the input layer may be the same as the number of nodes of the output layer, and the neural network may be a neural network of a type in which the number of nodes decreases and then, increases again from the input layer to the hidden layer. Further, in the neural network according to another embodiment of the present disclosure, the number of nodes of the input layer may be smaller than the number of nodes of the output layer, and the neural network may be a neural network of a type in which the number of nodes decreases from the input layer to the hidden layer. Further, in the neural network according to yet another embodiment of the present disclosure, the number of nodes of the input layer may be larger than the number of nodes of the output layer, and the neural network may be a neural network of a type in which the number of nodes increases from the input layer to the hidden layer. The neural network according to still yet another embodiment of the present disclosure may be a neural network of a type in which the aforementioned neural networks are combined.

A deep neural network (DNN) may refer to a neural network that includes a plurality of hidden layers in addition to the input and output layers. When the deep neural network is used, the latent structures of data may be determined. That is, latent structures of photos, text, video, voice, a protein sequence structure, a gene sequence structure, a peptide sequence structure, music (e.g., what objects are in the photo, what the content and feelings of the text are, what the content and feelings of the voice are), and/or a binding affinity between the peptide and MHC may be determined. The deep neural network may include a convolutional neural network (CNN), a recurrent neural network (RNN), an auto encoder, generative adversarial networks (GAN), a restricted Boltzmann machine (RBM), a deep belief network (DBN), a Q network, a U network, a Siam network, and the like. The description of the deep neural network described above is just an example and the present disclosure is not limited thereto.

As an example, the large language model of the present disclosure may mean a transformer-based artificial intelligence model. For example, the large language model may include a generative pre-trained transformer (GPT), or a bidirectional encoder representations from transformers (BERT).

The artificial intelligence-based large language model of the present disclosure may be expressed by a network structure with any structure, which includes the input layer, the hidden layer, and the output layer.

The neural network which may be used in the artificial intelligence based model of the present disclosure may be trained in at least one scheme of supervised learning, unsupervised learning, semi supervised learning, or reinforcement learning. The learning of the neural network may be a process in which the neural network applies knowledge for performing a specific operation to the neural network. As an example, a prediction model according to an embodiment of the present disclosure may be trained by a semi-supervised learning method that applies a mask to some amino acids among amino acid sequences and then matches the masked amino acids.

The neural network may be learned in a direction to minimize errors of an output. The learning of the neural network is a process of repeatedly inputting learning data into the neural network and calculating the output of the neural network for the learning data and the error of a target and back-propagating the errors of the neural network from the output layer of the neural network toward the input layer in a direction to reduce the errors to update the weights of each node of the neural network. In the case of the supervised learning, the learning data labeled with a correct answer is used for each learning data (i.e., the labeled learning data) and in the case of the unsupervised learning, the correct answer may not be labeled in each learning data. That is, for example, the learning data in the case of the supervised learning associated with the data classification may be data in which category is labeled in each learning data. The labeled learning data is input to the neural network, and the error may be calculated by comparing the output (category) of the neural network with the label of the learning data. As another example, in the case of the unsupervised learning associated with the data classification, the learning data as the input is compared with the output of the neural network to calculate the error. The calculated error is back-propagated in a reverse direction (i.e., a direction from the output layer toward the input layer) in the neural network and connection weights of respective nodes of each layer of the neural network may be updated according to the back propagation. A variation amount of the updated connection weight of each node may be determined according to a learning rate. Calculation of the neural network for the input data and the back-propagation of the error may constitute a learning cycle (epoch). The learning rate may be applied differently according to the number of repetition times of the learning cycle of the neural network. For example, in an initial stage of the learning of the neural network, the neural network ensures a certain level of performance quickly by using a high learning rate, thereby increasing efficiency and uses a low learning rate in a latter stage of the learning, thereby increasing accuracy.

In learning of the neural network, the learning data may be generally a subset of actual data (i.e., data to be processed using the learned neural network), and as a result, there may be a learning cycle in which errors for the learning data decrease, but the errors for the actual data increase. Overfitting is a phenomenon in which the errors for the actual data increase due to excessive learning of the learning data. For example, a phenomenon in which the neural network that learns a cat by showing a yellow cat sees a cat other than the yellow cat and does not recognize the corresponding cat as the cat may be an example of overfitting. The overfitting may act as a cause which increases the error of the machine learning algorithm. Various optimization methods may be used in order to prevent the overfitting. In order to prevent the overfitting, a method such as increasing the learning data, regularization, dropout of omitting a part of the node of the network in the process of learning, utilization of a batch normalization layer, etc., may be applied.

Disclosed is a computer readable medium storing the data structure according to an embodiment of the present disclosure. The above-described data structure may be stored in the storage unit in the present disclosure, executed by the processor, and transmitted and received by the communication unit.

The data structure may refer to the organization, management, and storage of data that enables efficient access to and modification of data. The data structure may refer to the organization of data for solving a specific problem (e.g., data search, data storage, data modification in the shortest time). The data structures may be defined as physical or logical relationships between data elements, designed to support specific data processing functions. The logical relationship between data elements may include a connection relationship between data elements that the user defines. The physical relationship between data elements may include an actual relationship between data elements physically stored on a computer-readable storage medium (e.g., persistent storage device). The data structure may specifically include a set of data, a relationship between the data, a function which may be applied to the data, or instructions. Through an effectively designed data structure, a computing device can perform operations while using the resources of the computing device to a minimum. Specifically, the computing device can increase the efficiency of operation, read, insert, delete, compare, exchange, and search through the effectively designed data structure.

The data structure may be divided into a linear data structure and a non-linear data structure according to the type of data structure. The linear data structure may be a structure in which only one data is connected after one data. The linear data structure may include a list, a stack, a queue, and a deque. The list may mean a series of data sets in which an order exists internally. The list may include a linked list. The linked list may be a data structure in which data is connected in a scheme in which each data is linked in a row with a pointer. In the linked list, the pointer may include link information with next or previous data. The linked list may be represented as a single linked list, a double linked list, or a circular linked list depending on the type. The stack may be a data listing structure with limited access to data. The stack may be a linear data structure that may process (e.g., insert or delete) data at only one end of the data structure. The data stored in the stack may be a data structure (LIFO—Last in First Out) in which the data is input last and output first. The queue is a data listing structure that may access data limitedly and unlike a stack, the queue may be a data structure (FIFO—First in First Out) in which late stored data is output late. The deque may be a data structure capable of processing data at both ends of the data structure.

The non-linear data structure may be a structure in which a plurality of data are connected after one data. The non-linear data structure may include a graph data structure. The graph data structure may be defined as a vertex and an edge, and the edge may include a line connecting two different vertices. The graph data structure may include a tree data structure. The tree data structure may be a data structure in which there is one path connecting two different vertices among a plurality of vertices included in the tree. That is, the tree data structure may be a data structure that does not form a loop in the graph data structure.

Throughout this specification, the large language model, the artificial intelligence-based model, the computation model, the neural network, the network function, and the neural network may be used as the same meaning. Hereinafter, the large language model, the artificial intelligence-based model, the computation model, the neural network, the network function, and the neural network will be integrated and described as the neural network. The data structure may include the neural network. In addition, the data structures, including the neural network, may be stored in a computer readable medium. The data structure including the neural network may also include data preprocessed for processing by the neural network, data input to the neural network, weights of the neural network, hyper parameters of the neural network, data obtained from the neural network, an activation function associated with each node or layer of the neural network, and a loss function for training the neural network. The data structure including the neural network may include predetermined components of the components disclosed above. In other words, the data structure including the neural network may include all of data preprocessed for processing by the neural network, data input to the neural network, weights of the neural network, hyper parameters of the neural network, data obtained from the neural network, an activation function associated with each node or layer of the neural network, and a loss function for training the neural network or a predetermined combination thereof. In addition to the above-described configurations, the data structure including the neural network may include other specific information that determines the characteristics of the neural network. In addition, the data structure may include all types of data used or generated in the calculation process of the neural network, and is not limited to the above. The computer readable medium may include a computer readable recording medium and/or a computer readable transmission medium. The neural network may be generally constituted by an aggregate of calculation units which are mutually connected to each other, which may be called nodes. The nodes may also be called neurons. The neural network is configured to include one or more nodes.

The data structure may include data input into the neural network. The data structure including the data input into the neural network may be stored in the computer readable medium. The data input to the neural network may include learning data input in a neural network learning process and/or input data input to a neural network in which learning is completed. The data input to the neural network may include preprocessed data and/or data to be preprocessed. The preprocessing may include a data processing process for inputting data into the neural network. Therefore, the data structure may include data to be preprocessed and data generated by preprocessing. The data structure is just an example and the present disclosure is not limited thereto.

The data structure may include weights of the neural network (weights and parameters may be used interchangeably in the present disclosure). In addition, the data structures, including the weight of the neural network, may be stored in the computer readable medium. The neural network may include a plurality of weights. The weight may be variable and the weight is variable by a user or an algorithm in order for the neural network to perform a desired function. For example, when one or more input nodes are mutually connected to one output node by the respective links, the output node may determine a data value output from an output node based on values input in the input nodes connected with the output node and the weights set in the links corresponding to the respective input nodes. The data structure is just an example and the present disclosure is not limited thereto.

As a non-limiting example, the weight may include a weight which varies in the neural network learning process and/or a weight in which neural network learning is completed. The weight which varies in the neural network learning process may include a weight at a time when a learning cycle starts and/or a weight that varies during the learning cycle. The weight in which the neural network learning is completed may include a weight in which the learning cycle is completed. Accordingly, the data structure including the weights of the neural network may include a data structure including the weights which vary in the neural network learning process and/or the weights in which neural network learning is completed. Accordingly, the above-described weight and/or combinations of weights are included in a data structure including the weights of a neural network. The data structure is just an example and the present disclosure is not limited thereto.

The data structure including the weights of the neural network may be stored in the computer-readable storage medium (e.g., memory, hard disk) after a serialization process. Serialization may be a process of storing data structures on the same or different computing devices and later reconfiguring the data structure and converting the data structure to a form that may be used. The computing device may serialize the data structure to send and receive data over the network. The data structure including the weights of the serialized neural network may be reconfigured in the same computing device or another computing device through deserialization. The data structure including the weights of the neural network is not limited to the serialization. Furthermore, the data structure including the weights of the neural network may include a data structure (for example, B-Tree, R-Tree, Trie, m-way search tree, AVL tree, and Red-Black Tree in a nonlinear data structure) to increase the efficiency in the operation while minimally using resources of the computing device. The above-described matter is just an example and the present disclosure is not limited thereto.

The data structure may include hyper-parameters of the neural network. In addition, the data structures, including the hyper-parameters of the neural network, may be stored in the computer readable medium. The hyper-parameter may be a variable which may be varied by the user. The hyper-parameter may include, for example, a learning rate, a cost function, the number of learning cycle iterations, weight initialization (for example, setting a range of weight values to be subjected to weight initialization), and Hidden Unit number (e.g., the number of hidden layers and the number of nodes in the hidden layer). The data structure is just an example and the present disclosure is not limited thereto.

As the network function for the prediction model according to an embodiment of the present disclosure, a transformer may be considered. As an example, the prediction model may operate based on the transformer. Such a prediction model may be operated using, for example, a recurrent neural network to which an attention algorithm is applied or a transformer to which the attention algorithm is applied.

In an embodiment, the transformer may be constituted by an encoder that encodes embedded data and a decoder that decodes the encoded data. The transformer may have a structure that receives a series of data, and outputs a series of data of different types through encoding and decoding steps. In an embodiment, a series of data may be processed in a form which is enabled to be computed by the transformer. A process of processing a series of data in the form which is enabled to be computed by the transformer may include an embedding process. Expressions such as a data token, an embedding vector, embedding token, etc. may refer to data embedded in a form which may be processed by the transformer.

In order for the transformer to encode and decode a series of data, encoders and decoders within the transformer may be processed using an attention algorithm. The attention algorithm may mean an algorithm that obtains a similarity of one or more keys for a given query, reflects the given similarity to a value corresponding to each key, and then calculates an attention value by weighting the values to which the similarity is reflected.

Depending on how the query, key, and value are set, various types of attention algorithms may be classified. For example, if an attention is obtained by setting the query, key, and value all the same, this may mean a self-attention algorithm. In order to process a series of input data in parallel, when a dimension of the embedding vector is reduced and the attention is obtained by obtaining individual attention heads for each divided embedding vector, this may mean a multi-head attention algorithm.

In an embodiment, the transformer may be constituted by modules that perform a plurality of multi-head self-attention algorithms or multi-head encoder-decoder algorithms. In an embodiment, the transformer may also include additional components other than the attention algorithm, such as an embedding layer, a normalization layer, a Softmax layer, etc. A method for constituting the transformer by using the attention algorithm may include a method disclosed in Vaswani et al., Attention Is All You Need, 2017 NIPS, which is incorporated herein by reference.

The transformer is applied to various data domains such as an embedded natural language, embedded sequence information segmented image data, and an audio waveform to convert a series of input data into a series of output data. In order to convert data with various data domains into a series of data that are enabled to be input to the transformer, the transformer may embed the data. The transformer may process additional data expressing a relative positional relationship or phase relationship between a series of input data. Alternatively, the series of input data may be embedded by additionally reflecting vectors expressing relative positional relationships or phase relationships between the input data to the series of input data. In one example, the relative positional relationship between a series of input data may include a word order within the natural language sentence, a relative positional relationship of respective segmented images, a temporal order of segmented audio waveforms, etc., but is not limited thereto. A process of adding information expressing a relative positional relationship or phase relationship between a series of input data may be referred to as positional encoding.

One example of a method for embedding data and transforming the embedded data by the transformer is disclosed in Dosovitskiy, et al., AN IMAGE IS WORTH 16×16 WORDS: TRANSFORMERS FOR IMAGE RECOGNITION AT SCALE, which is incorporated herein by reference.

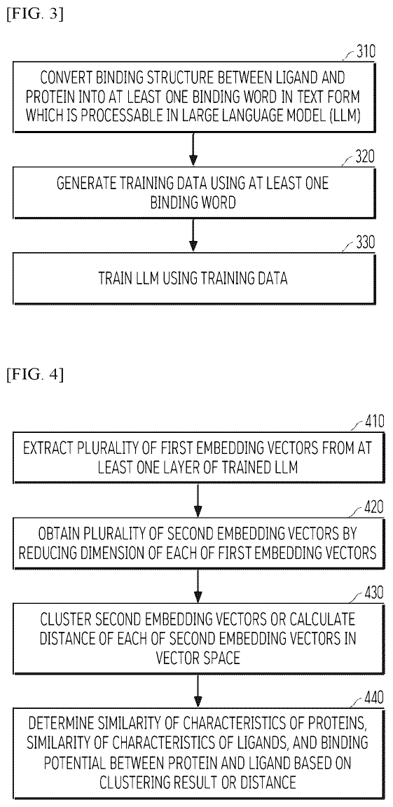

FIG. 3 exemplarily illustrates a method for training a large language model which processes a binding structure of a ligand and a protein according to an embodiment of the present disclosure.

In an embodiment, the steps illustrated in FIG. 3 may be performed by the computing device 100. In an additional embodiment, like a scheme in which some of the steps illustrated in FIG. 3 are performed by the user terminal and some other steps are performed by the server, the steps in FIG. 3 may also be implemented by a plurality of entities.

In an embodiment, the computing device 100 may transform a binding structure between a ligand and a protein into at least one binding word in a text format processable by a large language model (LLM) (310).

As an example, the ligand includes a material that may be bound to the protein as a substrate. In one example, the ligand may be a peptide. In one example, the ligand may be a hormone. In one example, the ligand may be a neurotransmitter. In one example, the ligand may be a cytokine. In one example, the ligand may be a growth factor. In one example, the ligand may be a signaling molecule. In one example, the ligand may be a drug comprising a pharmaceutical ingredient. In one example, the ligand may be a toxin. In addition to the examples described above, various biochemical substances may be included as an example of the ligand.

In the present disclosure, the protein may be used as a meaning of a receptor protein and a target protein which may be bound to the ligand. The target protein may be used to mean a protein that serves as a target substance for the ligand. The receptor protein may be used to mean a protein as a substance that accepts the ligand through binding. The protein, the target protein, the receptor, and the receptor protein may be used as the same meaning in this specification. In one example, the protein may be an enzyme. In one example, the protein may be a membrane protein. In one example, the protein may be a transmembrane protein. In one example, the protein may be an intracellular receptor. In one example, the protein may be a nuclear receptor. In addition to the examples described above, proteins having various structures and functions may be included as examples of proteins. Proteins that may be used in the present disclosure may be obtained through public databases (PDB, UniProt, and Pocketome). In addition, the proteins that may be used in the present disclosure may be obtained through a public dataset (CrossDocked2020).

As an example, identification information for the ligand may be obtained through the database. As an example, identification information for a protein-ligand complex may be obtained through the database.

In an embodiment, the proteins may be obtained from a database or dataset and used as pre-training data for training the large language model. As an example, a CrossDocked2020 dataset may be used to generate the pre-training data. As an example, the CrossDocked2020 dataset consists of binding structures of approximately 25000 proteins and pairs of ligands that are likely to be bound to each protein. Here, the total number of unique protein-ligand pairs may be approximately 600,000.

Respective protein-ligand pairs may have multiple binding poses. As an example, the binding pose may indicate a position and a three-dimensional structure of the ligand when the ligand is bound to the protein in the interaction between the protein and the ligand. In an embodiment, Convolutional Neural Networks (CNN) may be utilized to select a pose with a high binding force among 20 pose groups present in the CrossDocked2020 dataset. As an example, poses classified as having the high binding force through the CNN may be selected as a seed structure. As an example, the seed structure may be selected among structures that well reflect a binding state of the protein and the ligand.

As an example, each binding pose may be converted into a binding sentence form. As an example, each of the protein-ligand pairs may be identified by being assigned a protein-ligand complex ID. As an example, data converted into text formats of binding poses sharing the protein-ligand complex ID may constitute one binding paragraph.

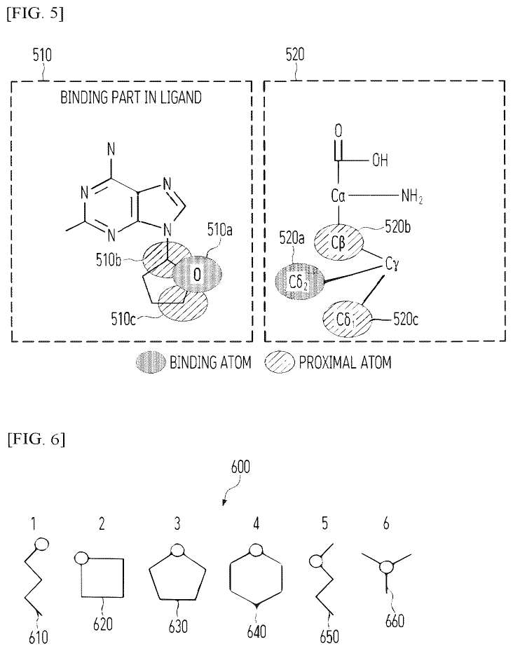

As an example, the binding structure between the ligand and the protein may include a structure of binding formed between a binding part of the ligand and a binding part of the protein. As an example, the binding structure may include a three-dimensional structure. As an example, the binding part of the protein may include protein residues that are positioned in a binding pocket. As an example, the binding structure between the ligand and the protein may include a structure related to an electron donor-acceptor interaction. Additional examples of the binding parts of the ligand and the protein are described later with regard to FIG. 5.

In an embodiment, the binding word may be one-dimensional data. In an embodiment, the binding word may represent a word in the form of binding of data from a perspective of the ligand and data from a perspective of the protein on the binding structure between the ligand and the protein. As an example, the binding word may be text-format data. As an example, the binding word may be a natural language. As an example, the binding word may include numbers. As an example, the binding word may include English letters. As an example, the binding word may include Greek letters. As an example, the binding word may include symbols. As another example, the binding word may be a combination selected from the group consisting of the English letters, the Greek letters, the numbers, and the symbols,

In an embodiment, the binding word may express a binding structure of the binding part of the ligand and a residue of the protein as data in a one-dimensional form. In an embodiment, the binding word may express an interaction between an electron donor and an electron acceptor between the binding part of the ligand and the residue of the protein.

In an embodiment, the binding word may be composed of a combination of multiple sub-binding words. In an embodiment, the binding word may include a first sub-binding word and a second sub-binding word.

In an embodiment, the first sub-binding word may express an interaction from a perspective of the ligand.

In an embodiment, the second sub-binding word may express an interaction from a perspective of the protein.

In an embodiment, the first sub-biding word and the second sub-binding word may constitute the binding word in a scheme in which the first sub-biding word and the second sub-binding word are concatenated with each other. As an example, concatenation may mean joining of multiple text data. As an example, the concatenation may mean that character strings are connected to each other in series. Through the concatenation, an end of word A and a beginning of word B may be connected. In an embodiment of the present disclosure, a three-dimensional structure between the ligand and the protein may be expressed by concatenating the first sub-binding word which expresses the interaction from a perspective of the ligand and the second sub-binding word which expresses the interaction from a perspective of the protein with each other.

In an embodiment, each of the first-sub binding word and/or the second sub-binding word may include sub-parts or sub-information.

In an embodiment, the first sub-binding word may include a first part expressing whether a role of the binding atom of the ligand is the electron donor or the electron acceptor on the binding structure, a second part identifying the binding atom of the ligand on the binding structure, and/or a third part identifying at least one proximal atom located proximal to the binding atom of the ligand on the binding structure. Here, the binding atom may be an atom which directly participates in the binding by serving as the electron donor or the electron acceptor. Here, the proximal atom may be one or more atoms located proximal to the binding atom in the ligand. As an example, the proximal atom may mean one or more atoms located less than a predetermined threshold distance from the binding atom in the ligand. As an example, the proximal atom may mean one or more atoms directly connected to the binding atom in the ligand. As an example, the proximal atom may mean an atom which exists at a location closest to the binding atom in the ligand. As an example, the proximal atom may mean atoms of a predetermined number in an order closest to the binding atom in the ligand.

In another embodiment, the first sub-binding word may be a form in which first information expressing whether the role of the binding atom of the ligand is the electron donor or the electron acceptor on the binding structure, second information identifying a binding form formed by the binding atom of the ligand with other atoms of the ligand on the binding structure, third information identifying a first proximal atom located proximal to the binding atom of the ligand, and identifying a binding form formed by the first proximal atom with other atoms of the ligand on the binding structure, fourth information identifying the binding atom of the ligand and identifying a binding form formed by the binding atom of the ligand with other atoms of the ligand, on the binding structure, and/or fifth information identifying a second proximal atom located proximal to the binding atom of the ligand, and identifying a binding form formed by the second proximal atom with other atoms of the ligand, on the binding structure are concatenated with each other. Here, the binding atom may be an atom which directly participates in the binding by serving as the electron donor or the electron acceptor. Here, the proximal atom may be one or more atoms located proximal to the binding atom in the ligand, and an example of the proximal atom will be replaced with the example described above.

A binding form formed by a specific atom with other atoms will be described later with regard to FIG. 6.

In an embodiment, the second sub-binding word may include a first part identifying a binding amino acid of the protein on the binding structure, a second part identifying a receptor binding atom obtained from the binding amino acid, and/or a third part identifying at least one receptor proximal atom located proximal to the receptor binding atom. Here, the binding amino acid may be an amino acid type of the protein residue involved in the binding. For example, alanine, arginine, asparaginic acid, aspartic acid, cysteine, glutamic acid, glutamine, glycine, histidine, isoleucine, leucine, lysine, methionine, phenylalanine, proline, serine, threonine, tryptophan, and valine may correspond to binding amino acids. Here, the receptor binding atom may be an atom that is directly involved in the binding by serving as the electron donor or the electron acceptor in the protein. Here, the receptor proximal atom may be one or more atoms located proximal to the receptor binding atom in the protein. As an example, the receptor proximal atom may mean one or more atoms located less than a predetermined threshold distance from the receptor binding atom in the protein. As an example, the receptor proximal atom may mean one or more atoms directly connected to the receptor binding atom in the protein. As an example, the receptor proximal atom may mean an atom that exists at a location closest to the receptor binding atom in the protein. As an example, the receptor proximal atom may mean atoms of a predetermined number in an order closest to the receptor binding atom in the protein.

In an embodiment, the first sub-binding word and the second sub-binding word constituting the binding word are concatenated with each other through a first expression to express the binding word. As an example, the first expression may include a hyphen or a dash. Parts or information constituting the first sub-binding word, and parts or information constituting the second sub-binding word are concatenated with each other through a second expression to express the sub-binding word. As an example, the second expression may include an underbar, or a space. By configuring the first and second expressions differently as described above, the sub-binding words may be distinguished within the binding word, and the parts in the sub-binding word may be distinguished.

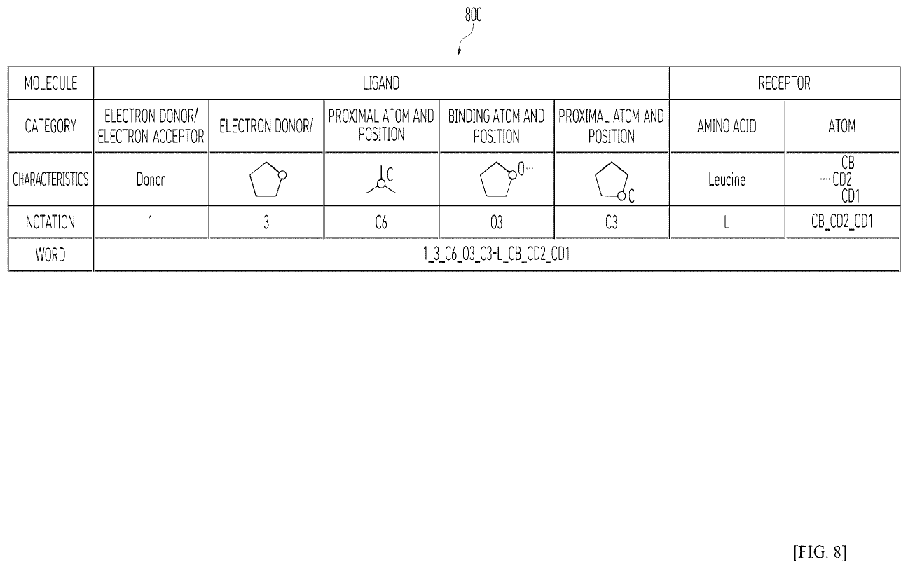

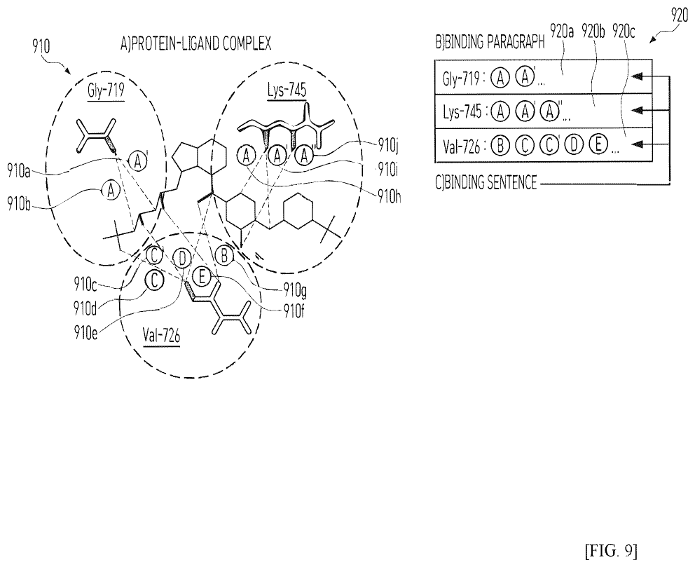

Additional examples for the binding word will be described later with regard to FIGS. 8 and 9.

In an embodiment, the computing device 100 may generate training data by using at least one binding word (320).

In an embodiment, an artificial intelligence model may require diverse training data to operate effectively in different situations.

As an example, the training data may include one-dimensional data in order to train the LLM. As an example, the training data may include text data. As an example, the training data may include the binding structure between the ligand and the protein converted into the text format. As an example, the training data may include the binding word. As an example, the training data may include a binding sentence corresponding to a set of binding words. As an example, the training data may include a binding paragraph corresponding to a set of binding sentences. As an example, the training data may include data annotated to the binding structure converted into the text format.

In an embodiment, the training data may be generated by using the binding word. In an embodiment, the computing device 100 may generate a binding sentence expressing a binding structure between one protein residue and all ligand fragments bound thereto by combining a plurality of binding words, and the training data may be generated by using the binding sentence.

In an embodiment, the training data may be generated by using the binding sentence. In an embodiment, the computing device 100 may generate a binding paragraph expressing a binding structure between one protein binding pocket and all ligand fragments bound thereto by combining a plurality of binding sentences, and the training data may be generated by using the binding paragraph.

In an embodiment, the binding sentence may be constituted by combining a plurality of binding words. As an example, the binding sentence may express a binding structure between a single protein residue and all ligand fragments that are bound to the protein residue. Here, the ligand fragment may be three atoms 510a, 510b, and 510c including a binding atom involved in the binding in the binding part of the ligand and two proximal atoms located proximal to the binding atom. The ligand fragments will be described later with regard to FIG. 5.

In an embodiment, the binding paragraph may be constituted by combining the plurality of binding sentences. As an example, the binding paragraph may express a binding structure between the single protein binding pocket and each of all ligand fragments that are bound to the binding pocket.

As an example, annotation may be a process of labeling or annotating data for training the artificial intelligence model. According to an embodiment of the present disclosure, data that may be annotated to the binding structure converted into the text format may include protein identification information, species information, and ligand identification information.

Here, the protein identification information may be a protein ID disclosed through the protein database. As an example, the protein database may include PDB and UniProt.

Here, the species information may be species information of the protein disclosed through the protein database. As an example, even if proteins perform the same function within a living organism, sequences of proteins found in different species may be different. As an example, the species information may include human beings.

Here, the ligand identification information may be information on the ligand which may be bound to each protein disclosed through the protein database. As an example, the ligand identification information may be a ligand ID disclosed through the protein database.

In an embodiment, the computing device 100 may train the LLM using the training data (330).

As an example, the step of training the LLM by using the training data may include a step of tokenizing the training data. As an example, the tokenization may include word-based tokenization in which each word serves as one token to form a vocabulary, and byte-pair encoding tokenization in which all words included in respective binding paragraphs are connected to a space character, and the respective binding paragraphs are distinguished by a newline character. At this time, the vocabulary may be a set of all words used to train the model. For example, when the vocabulary is generated by tokenizing texts such as ‘I like peaches. He raises a cat.’, words such as ‘I’, ‘peaches’, ‘like’, ‘he’, ‘a’, ‘cat’, and ‘raises’ may be included in the vocabulary.

As an example, a tokenizer may be used for the tokenization. As an example, a BPE tokenizer may be used in order to perform the byte-pair encoding tokenization. As an example, special tokens may be used for the tokenization. As an example, the special tokens may include [PAD], [September], and [UNK]. As an example, the tokenization may use the [PAD] token to ensure that a length of the token meets a predetermined criterion. As an example, the tokenization may use the [September] token to recognize a boundary of each sentence by indicating a beginning and an end of each sentence. As an example, the tokenization may use the [UNK] token to replace a character string that does not exist in the model's vocabulary.

In an embodiment, each of the tokens generated by tokenization may correspond to the binding word. In an embodiment, the respective tokens generated by tokenization may correspond to the sub-binding words constituting the binding word. In an embodiment, the respective tokens generated by tokenization may correspond to the parts or information constituting the sub-binding word.

The step of training the LLM in the present disclosure may mean pre-training the artificial intelligence model using a large-scale text dataset including the converted binding words as the training data according to an embodiment of the present disclosure. As an example, the large-scale text dataset here may include a natural language processing (corpus) dataset.

The step of training the LLM in the present disclosure may include re-training or additionally training the pre-trained LLM using training data (e.g., training data including binding words) required for a specific task or domain (e.g., a bio domain, or a domain related to the interaction between the ligand and the protein).

The step of training the LLM in the present disclosure may be operated in a scheme of transfer learning to transfer knowledge of a pre-trained artificial intelligence model to another task or domain (e.g., a bio domain, or a domain related to the interaction between the ligand and the protein).

FIG. 4 exemplarily illustrates a method using an embedding vector obtained from the trained large language model according to an embodiment of the present disclosure.

In an embodiment, the steps illustrated in FIG. 4 may be performed by the computing device 100. In an additional embodiment, like a scheme in which some of the steps illustrated in FIG. 4 are performed by the user terminal and some other steps are performed by the server, the steps in FIG. 4 may also be implemented by a plurality of entities.

In an embodiment, the computing device 100 may extract a plurality of first embedding vectors from at least one layer of the trained LLM (410).

As an example, in the LLM, vector embedding may mean mapping a text or word to a high-dimensional vector space. Words with similar meaning may exist at positions close to each other as the words are embedded in the vector space. Accordingly, a semantic similarity between words may be calculated using the embedding vector. The embedding vector may be used to cluster and visualize words or sentences having similar characteristics. The embedding vector may be used for analyzing a relationship between words. The embedding vector may be utilized for a text generation function of the LLM. In such an example, the LLM may generate a next word of an input word, and complete a sentence by using the embedding vector.

As an example, the embedding vector may be used to numerically express the binding word or token. In the large language model, the embedding vector may be used for expressing the semantic similarity of the words as a spatial distance. Throughout this specification, the embedding vector may be used for expressing the similarity of the binding structure converted into the text format as the spatial distance.

In an embodiment, the computing device 100 may extract an embedding vector from at least one layer of the LLM trained (or pre-trained) according to the training scheme in FIG. 3. As an example, the embedding vector may be generated and extracted in response to a word input into the LLM.

In an embodiment, the computing device 100 may obtain a plurality of second embedding vectors by reducing dimension of respective first embedding vectors (420).

Reducing the dimension of the embedding vector may mean a process of compressing the high-dimensional embedding vector to a low-dimensional vector. As an example, reducing the dimension of the embedding vector may accelerate a training and/or inference speed of a model by reducing the amount of computation during the inference and/or training process of the model. As an example, reducing the dimension of the embedding vector may facilitate data visualization and interpretation. The data visualization and interpretation in this specification may include a clustering technique. As an example, as a method for reducing the dimension of the embedding vector, dimension reduction algorithms such as UMAP, principal component analysis (PCA), and t-Stochastic Neighbor Embedding (t-SNE) may be used. In an embodiment, a second embedding vector may be obtained by reducing dimensions of high-dimensional first embedding vectors.

In an embodiment, the computing device 100 may cluster the second embedding vectors or calculate distances between the respective second embedding vectors in the vector space (430).

In an embodiment, the clustering may mean grouping embedding vectors, each with unique characteristics, into groups with similar characteristics. As an example, Louvain clustering, K-means, DBSCAN, and Hierarchical clustering may be used as clustering algorithms.

In an embodiment, the computing device 100 may determine a similarity of characteristics of proteins, a similarity of characteristics of ligands, and a binding probability of the protein and the ligand based on a result of the clustering or the distance (440).

As an example, the characteristics may include structural characteristics, functional characteristics, and genetic characteristics.

As an example, characteristics of proteins may include structural characteristics of the proteins.

As an example, BLAST or FASTA may be used to analyze a similarity of structural characteristics of proteins. Alternatively, DALI or CE may be used to analyze the similarity of the structural characteristics of the proteins. As an example, the characteristics of the proteins may include the functional characteristics of the proteins. As an example, proteins with similar functional characteristics may have similar physiological functions.

As an example, the characteristics of the proteins may include the genetic characteristics of the proteins. As an example, proteins with similar genetic characteristics may be included in a homologous gene or analogous gene.

As an example, the characteristics of the ligands may include structural characteristics of the ligands. As an example, DALI or CE, which are a structural comparison tool provided in RCSB PDB, may be used to analyze a similarity of the structural characteristics of the ligands. As an example, a similarity index may be calculated to numerically express the similarity of the structural characteristics of the ligands. The similarity index may be an index that may quantify and compare a structural similarity between two ligands. As an example, the similarity index may include Root Mean Square Deviation (RMSD) or Tanimoto coefficient.

As an example, the characteristics of the ligands may include functional characteristics of the ligands. As an example, the functional characteristics of the ligands may mean a biological activity that each ligand possesses. As an example, to numerically compare the functional characteristics of the ligands, an EC50 (effective concentration 50%) or IC50 (inhibitory concentration 50%) value may be obtained through experiment.

According to an embodiment of the present disclosure, the similarity of the characteristics of the ligands is used to determine additional uses of drugs corresponding to the ligands.

As an example, the binding potential between the protein and the ligand may be determined by analyzing the similarity of the characteristics of the proteins and/or the similarity of the characteristics of the ligands.

As an example, the examples above may be used for the computing device 100 to validate a similarity and/or a binding potential determined based on the result of the clustering or the distance according to the method of the present disclosure.

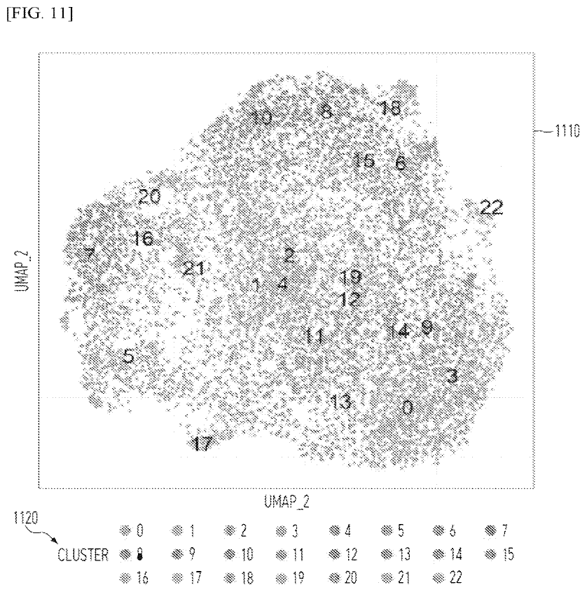

An embodiment using clustering is described later with regard to FIG. 11 or below.

FIG. 5 illustrates a binding structure including a binding part in the ligand and a protein residue that is target of a conversion according to an embodiment of the present disclosure.

In an embodiment, a binding part 510 in the ligand, as a part of the ligand, may be a part that forms binding with a protein residue 520 involved in binding within the binding pocket of the protein. As an example, the binding part in the ligand may include a binding atom 510a. In FIG. 5, an electron donor-electron acceptor interaction between the binding atom 510a in the ligand and a binding atom 520a in the protein residue is represented by dotted lines. As an example, a binding atom in the protein residue bound to the binding atom 510a in the ligand may be a receptor binding atom 520a. As an example, atoms located proximal to the binding atom within the binding part in the ligand and the protein residue may be proximal atoms. As an example, there may be two proximal atoms, one in the binding part in the ligand and one in the protein residue. As an example, proximal atoms 520b and 520c in the protein residue may be referred to as receptor proximal atoms, as distinguished from proximal atoms 510b and 510c in the ligand.

As an example, an nomenclature of organic chemistry may be used to distinguish a plurality of carbon atoms present in a single protein residue 520. As an example, in the protein residue 520, carbon directly connected to a carboxyl group (COOH) may be defined as alpha carbon (Cα). As an example, in the protein residue 520, the other carbons connected to the alpha carbon may be defined as beta carbon (Cβ), gamma carbon (Cγ), and delta carbon (Cδ), in order. As an example, in the case of leucine 520 of FIG. 5, two delta carbons connected to the gamma carbon may be defined as first delta carbon (Cδ¹) and second delta carbon (Cδ²), respectively.

As an example, the receptor binding atom may be the second delta carbon (Cδ²) (520a). As an example, the receptor proximal atoms may be beta carbon (Cβ) and the first delta carbon (Cδ₁) (520b and 520c). The nomenclature of organic chemistry may also be used to distinguish a plurality of other elements in addition to carbon atoms.

As an example, the binding atom and proximal atoms 510a, 510b, and 510c in the binding part within the ligand may be referred to as ligand fragments. As an example, the binding atom and proximal atoms 520a, 520b, and 520c in the protein residue may be referred to as residue fragments.

A technique according to an embodiment of the present disclosure may perform preprocessing into binding words for training and/or inference of the LLM by converting the binding part in the ligand exemplified in FIG. 5, into first sub-binding words and converting bindings of residues in the protein into second sub-binding words.

FIG. 6 exemplarily illustrates a binding form in which a binding atom or a proximal atom of the ligand, or a receptor binding atom or proximal atom forms with other atoms within the ligand or the receptor according to an embodiment of the present disclosure. In an embodiment, a binding form 600 which an atom forms with other atoms include a total of six types 610 to 660 as an example and not a limitation.

In a first binding form, the binding atom or proximal atom may be located at an end of a linearly bound atomic structure (610).

In a second binding form, the binding atom or proximal atom may be one of the four atoms bound in a ring structure (620).

In a third binding form, the binding atom or proximal atom may be one of the five atoms bound in the ring structure (630).

In a fourth binding form, the binding atom or proximal atom may be one of the six atoms bound in the ring structure (640).

In a fifth binding form, the binding atom or proximal atom may be located at a second position from the distal end of the linearly bound atomic structure (650).

In a sixth binding form, the binding atom or proximal atom may have a binding structure in which the binding atom or proximal atom is surrounded by other atoms or structures (660).

As an example, the first to sixth binding forms may be included in the first sub-binding word as the second information.

As an example, the first to sixth binding forms may be included in the third information, the fourth information, and/or the fifth information within the first sub-binding word to indicate the binding form formed by the binding atom or proximal atom with other atoms.

As an example, the first to sixth binding forms may be included in the second part or the third part within the second sub-binding word to indicate a binding form formed by the receptor binding atom or acceptor proximal atom with other atoms.

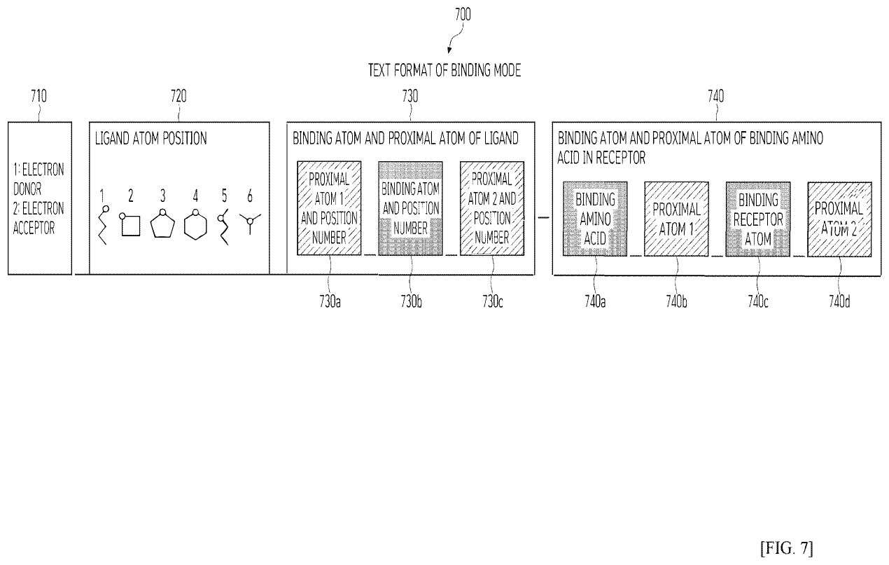

FIG. 7 exemplarily illustrates a conversion to a text format processable in the large language model of the binding structure of the ligand and the protein according to an embodiment of the present disclosure.

In an embodiment, FIG. 7 may conceptually illustrate conversion of the binding structure of the ligand and the protein into the binding word (700).

In an embodiment, the binding word 700 may include first sub-binding words 710, 720, and 730 and a second sub-binding word 740.

In an embodiment, the first sub-binding words may be parts 710, 720, and 730 that express the interaction from a perspective of the ligand.

In an embodiment, the second sub-binding word may be parts 740 that expresses the interaction from a perspective of the protein.

In an embodiment, the first sub-binding words 710, 720, and 730 and the second sub-binding word 740 may constitute the binding word 700 in a scheme in which the first sub-binding words 710, 720, and 730 and the second sub-binding word 740 are concatenated with each other (e.g., in a scheme in which one end of each is connected in series).

In an embodiment, the first sub-binding word may be a form in which first information 710 expressing whether the role of the binding atom of the ligand is the electron donor or the electron acceptor on the binding structure, second information 720 identifying a binding form formed by the binding atom of the ligand with other atoms of the ligand on the binding structure, third information 730a identifying a first proximal atom located proximal to the binding atom of the ligand, and identifying a binding form formed by the first proximal atom with other atoms of the ligand on the binding structure, fourth information 730b identifying the binding atom of the ligand and identifying a binding form formed by the binding atom of the ligand with other atoms of the ligand on the binding structure, and/or fifth information 730c identifying a second proximal atom located proximal to the binding atom of the ligand, and identifying a binding form formed by the second proximal atom with other atoms of the ligand on the binding structure are concatenated with each other.

As an example, the first information may be represented as a number. As an example, the first information may be represented as character. As an example, the first information may be represented as a symbol. As an example, the first information may be displayed in any scheme of a text form belonging to one-dimensional data.

As an example, the second information may be displayed in a scheme to identify the binding forms 610 to 660 illustrated in FIG. 6. As an example, the second information may be represented as a number. As an example, the second information may be represented as a character. As an example, the second information may be represented as a symbol. As an example, the second information may be displayed in any scheme of the text form belonging to one-dimensional data.

As an example, the third to fifth information may be represented in a scheme to identify the binding atoms or proximal atoms and binding forms thereof. As an example, element symbols may be used to express the binding atom or proximal atom. As an example, additional symbols or characters may be used to identify different atoms of the same element. As an example, the binding forms illustrated in FIG. 6 may be used to identify the binding form of the binding atom or proximal atom. As an example, any scheme of the text form belonging to one-dimensional data adopted to display the second information 720 may be reused to display the third to fifth information.

In an embodiment, the second sub-binding word may include a first part 740a identifying the binding amino acid of the protein on the binding structure, a second part 740c identifying the receptor binding atom obtained from the binding amino acid, and third parts 740b and 740d identifying at least one receptor proximal atom located proximal to the receptor binding atom.

As an example, the first part may be represented in a scheme to identify the type of amino acid. As an example, the first part may be represented by a one letter code for the amino acid. As an example, the first part may be represented by a three letter code for the amino acid.

As an example, the second part and the third part may be represented in a scheme to identify the binding atoms or proximal atoms. As an example, element symbols may be used to express the binding atom or proximal atom. As an example, additional symbols or characters may be used to identify different atoms of the same element. For example, to distinguish multiple carbon atoms 510a, 510b, and 510ccontained in a single protein residue 520, expressions such as CDB, CD1, and CD2 may be used as in FIG. 8.

In an embodiment, the first sub-binding word and the second sub-binding word constituting the binding word are concatenated with each other through a first expression to express the binding word. As an example, the first expression may include symbols or characters that may be represented in the text form.

In an embodiment, parts or information constituting the first sub-binding word are concatenated with each other through the second expression to express the first sub-binding word.

In an embodiment, parts or information constituting the second sub-binding word are concatenated with each other through the second expression to express the second sub-binding word. As an example, the second expression may include symbols or characters that may be represented in the text form.

In an embodiment, the first expression and the second expression may be different from each other. For example, the first expression may take a form of a hyphen, a dash, and/or a parenthesis, and the second expression may take a form of an underbar, a space, and/or a comma.

FIG. 8 additionally illustrates a conversion to a text format processable in the large language model of the binding structure of the ligand and the protein according to an embodiment of the present disclosure.

According to an embodiment of the present disclosure, the example illustrated in FIG. 8 may be one example of converting an exemplary binding structure of binding parts of the ligand and the protein into the binding word of the text format. As an example, the example 800 of FIG. 8 may include a form in which data converted into text formats of binding structures corresponding to the binding part 510 in ligand and the protein residue 520 are concatenated with each other. Through the following contents, a method which converts the exemplary structure illustrated in FIG. 5 into the exemplary binding word illustrated in FIG. 8 will be described.

In an embodiment, the text converted data corresponding to the binding part 510 in ligand may be the first sub-binding word. In an embodiment, the text converted data corresponding to the protein residue 520 may be the second sub-binding word. In an embodiment, the first sub-binding word and the second sub-binding word are concatenated with each other to form the binding word through the first expression. In an embodiment, the first expression may be a hyphen or a dash, as in FIG. 8 (800).

In an embodiment, the first sub-binding word may be a form in which the first information 710, the second information 720, the third information 730a, the fourth information 730b, and/or the fifth information 730c are concatenated with each other. In an embodiment, the information constituting the first sub-binding word may be concatenated through the second expression. In an embodiment, the second expression may be the underbar as in FIG. 8 (800).

In an embodiment, the first information 710 may be represented in any scheme of the text form belonging to one-dimensional data, such as whether the bonding atom 510a is the electron donor or the electron acceptor on the binding structure 510 and 520. As an example, the first information may be represented as one of two symbols, one of two characters, or one of two numbers. As an example, the first information 710 may be represented as 1 when the ligand binding atom acts as the electron donor. As an example, the first information 710 may be represented as 2 when the ligand binding atom acts as the electron acceptor. As an example, the ligand binding atom 510a of FIG. 5 acts as the electron donor in the binding structure, and therefore the first information 710 of the bonding word may be 1.

In an embodiment, in the second information 720, a binding form formed by the binding atom 510a with other atoms of the ligand may be represented in any scheme of the text form belonging to one-dimensional data. As an example, when six binding forms are assumed as in FIG. 6 (600), the second information may be represented as numbers, symbols, and/or characters corresponding to respective binding forms. As an example, the ligand binding atom 510a of FIG. 5 is one of five atoms bound in the ring structure (630). Such a binding form may be a third form illustrated in FIG. 6, and therefore, as an example and not a limitation, the second information 720 of the binding word may be 3.

In an embodiment, the third information 730a may represent a binding form formed by the first proximal atom 510b of the ligand with other atoms of the ligand in a scheme to identify an atom and a binding form of the atom. As an example, the first proximal atom 510b of the ligand may be represented as an element symbol. As an example, the first proximal atom 510b of FIG. 5 may be represented as an element symbol C, as carbon. As an example, the third information may include a part which represents the binding form formed by the first proximal atom 510b with other atoms of the ligand in any scheme of the text form belonging to one-dimensional data. As an example, the scheme may be a scheme which is the same as the scheme of representing the second information 720. As an example, if six binding forms are assumed as in FIG. 6 (600), the binding form formed by the first proximal atom 510b with other atoms of the ligand may be represented as numbers, symbols, and/or characters corresponding to respective binding forms. As an example, the first proximal atom 510b of FIG. 5 has a binding structure surrounded by other atoms or structures (660). Such a binding form may be a sixth form illustrated in FIG. 6, and therefore, as an example and not a limitation, may have a binding form corresponding to 6. Therefore, as an example, the third information 730a may include C corresponding to identification information of the first proximal atom and 6 corresponding to the information of the binding form of the first proximal atom. As an example, the third information may be C6.

In an embodiment, the fourth information 730b may represent a binding form formed by the binding atom of the ligand with other atoms of the ligand in a scheme to identify an atom and a binding form of the atom. As an example, the ligand binding atom 510a may be represented as the element symbol. As an example, the ligand binding atom 510a of FIG. 5 as oxygen may be represented as an element symbol O. As an example, the fourth information may include a part which represents the binding form formed by the ligand binding atom 510a with other atoms of the ligand in any scheme of the text form belonging to one-dimensional data. As an example, the scheme may be a scheme which is the same as the scheme of representing the second information 720. As an example, if six binding forms are assumed as in FIG. 6 (600), the binding form formed by the ligand binding atom 510a with other atoms of the ligand may be represented as numbers, symbols, and/or characters corresponding to respective binding forms. As an example, the ligand binding atom 510a of FIG. 5 is one of five atoms bound in the ring structure (630). Such a binding form may be a third form illustrated in FIG. 6, and therefore, as an example and not a limitation, may have a binding form corresponding to 3. Therefore, as an example, the fourth information 730b may include O corresponding to information of the ligand binding atom and 3 corresponding to the information of the binding form of the ligand binding atom. As an example, the fourth information may be O3.

In an embodiment, the fifth information 730c may represent a binding form which the second proximal atom 510c of the ligand forms with other atoms of the ligand in a scheme to identify an atom and a binding form of the atom. As an example, the second proximal atom 510c of the ligand may be represented as the element symbol. As an example, the second proximal atom 510c of FIG. 5 may be represented as the element symbol C, as carbon. As an example, the fifth information may include a part which represents the binding form formed by the second proximal atom 510c with other atoms of the ligand in any scheme of the text form belonging to one-dimensional data. As an example, the scheme may be a scheme which is the same as the scheme of representing the second information 720. As an example, if six binding forms are assumed as in FIG. 6 (600), the binding form which the second proximal atom 510c forms with other atoms of the ligand may be represented as numbers, symbols, and/or characters corresponding to respective binding forms. As an example, the second proximal atom 510c of FIG. 5 is one of five atoms bound in the ring structure (630). Such a binding form may be a third form illustrated in FIG. 6, and therefore, as an example and not a limitation, may have a binding form corresponding to 3. Therefore, as an example, the fifth information 730c may include C corresponding to information of the second proximal atom and 3 corresponding to the information of the binding form of the second proximal atom. As an example, the fifth information may be C3.

In an embodiment, the information constituting the first sub-binding word may be concatenated through the second expression. As an example, if the second expression is the underbar, the information of the first sub-binding word may be concatenated through the underbar as follows: 1_3_C6_O3_C3.

In an embodiment, the second sub-binding word may be a form in which the first part 740a, the second part 740c, and/or the third parts 740b and 740d are concatenated with each other. In an embodiment, the parts constituting the second sub-binding word may be concatenated through the second expression. In an embodiment, the second expression may be the underbar as in FIG. 8 (800).

In an embodiment, the first part 740a may be represented in a scheme to identify the type of amino acid corresponding to the protein residue 520 within the binding structure. As an example, the first part may be represented by the one letter code for the amino acid. As an example, the first part may be represented by the three letter code for the amino acid. As an example, the protein residue 520 in FIG. 5 is leucine, and therefore the first part of the second sub-binding word may be L, a one letter code for leucine.

In an embodiment, the second part 740c may be represented in a scheme to identify the receptor binding atom obtained from the protein residue 520. As an example, the receptor binding atom 520a of the protein residue may be represented as the element symbol. As an additional example, to distinguish multiple carbon atoms 510a, 510b, and 510c contained in the single protein residue 520, a nomenclature of organic chemistry may be used. As an additional example, carbon atoms distinguished by the nomenclature of organic chemistry may be converted into a simpler scheme to form the training data for the artificial intelligence model. As an example, expressions such as CB, CD1, and CD2 may be used as in FIG. 8. As an example of the simpler method above, subscripts or superscripts may be converted to non-subscript numbers. As an example, Greek letters may be converted to English uppercase or lowercase. As an example of the simpler method, alpha (a) may be converted to an uppercase A. Similarly, beta (B) may be converted to an uppercase B. As an example, the receptor binding atom 520a may be referred to as the second delta carbon (Cδ₂) by the nomenclature of organic chemistry. As an example, the second delta carbon may be represented in a form such as CD2 to constitute the second sub-binding word 740 as a second part 740c. As an example, the second part may be CD2.

In an embodiment, the third parts 740b and 740d may be represented in a scheme to identify the receptor proximal atoms 520b and 520c obtained from the protein residue 520. As an example, the receptor proximal atoms 520b and 520c of the protein residue may be represented as the element symbols.

As an additional example, in addition to the element symbol, the nomenclature of organic chemistry described above may be used to distinguish a plurality of carbon atoms which exist within the single protein residue 520. As an additional example, the plurality of carbon atoms distinguished by the nomenclature may be converted in a simpler scheme. The examples for the nomenclature and the simple scheme are replaced with those described above.

As an example, the conversion may be used to generate the training data for the artificial intelligence model. As an example, the conversion may be used to constitute the binding word. As an example, the conversion may be used to constitute the first sub-binding word or the second sub-binding word.

As an example, the first receptor proximal atom 520b may be referred to as the beta carbon (Cβ) by the nomenclature of organic chemistry. As an example, the beta carbon may be represented in a form such as CB and may constitute a part 740b of the third part.

As an example, the second acceptor proximal atom 520c may be referred to as the first delta carbon (Cδ¹) by the nomenclature of organic chemistry. As an example, the first delta carbon may be represented in a form such as CD1 and may constitute a part 740d of the third part.

In an embodiment, the parts or information constituting the second sub-binding word may be concatenated through the second expression. As an example, if the second expression is the underbar, the parts of the second sub-binding word may be concatenated through the underbar as follows: L_CB_CD2_CD1.

In an embodiment, the first sub-binding word and the second sub-binding word constituting the binding word are concatenated with each other through a first expression to express the binding word. As an example, if the first expression is the hyphen, the first sub-binding word and the second sub-binding word may be concatenated through the hyphen as follows: 1_3_C6_03_C3-L_CB_CD2_CD1.

The example in FIG. 8 is only one example of converting the binding structure of the protein and the ligand into the text form according to the technique disclosed herein. The binding word may be configured to take on more diverse forms by adopting the schemes of representing the information and/or parts of the first sub-binding word and the second sub-binding word differently, and by adopting the first expression and/or the second expression differently.

FIG. 9 illustrates an example of a binding word, a binding sentence, and a binding paragraph which convert the binding structure of the ligand and the protein to the text form processable in the large language model of according to an embodiment of the present disclosure.

In an embodiment, the binding word may correspond to a binding structure formed by a single ligand fragment and a single residue fragment. Accordingly, even within a single protein-ligand complex 910, there may be a plurality of separate binding words depending on the protein residue, depending on the binding atom within the protein residue, and/or depending on the binding part within the ligand, depending on the binding atom within the ligand.

An exemplary protein-ligand complex is illustrated through FIG. 9 (910). Gly-719, Lys-745 and Val-726 included in the complex may all be considered as protein residues present in the binding pocket within a single protein.

In an embodiment, two different bindings 910a and 910b between Gly-719 and the ligand may be converted into separate words. As an example, the bindings 910a and 910b may share a single receptor binding atom, but their respective ligand binding atoms and ligand proximal atoms may be different. Accordingly, the bindings 910a and 910b may be bindings between a single residue and separate ligand fragments. As an example, it may be considered that binding words according to the conversion of the present disclosure of the bindings 910a and 910b may share some of or all the second sub-binding words, while the binding words will have different first sub-binding words.

In an embodiment of the present disclosure, the binding sentence may express a binding between the single protein residue and all ligand fragments that are bound to the single protein residue. The binding sentence may be generated by combining a plurality of binding words. As an example, the bindings formed between Gly-719 and the ligand within the protein-ligand complex may be expressed as a single binding sentence. As an example, the bindings formed between Gly-719 and the ligand may be expressed as a plurality of binding words 910a and 910b, and the plurality of binding words may be expressed as a single binding sentence 920a.

Further, bindings between Lys-745 and the ligand, between Val-726 and the ligand are illustrated in FIG. 9.

As an example, in Val-726, bindings 910c, 910d, and 910e may be bindings formed by a single binding atom within the single protein residue with different binding atoms within a single ligand, similar to the bindings 910a and 910b. As an example, the bindings 910c, 910d, and 910e may share a single receptor binding atom, but their respective ligand binding atoms and ligand proximal atoms may be different. Accordingly, the bindings 910c, 910d, and 910e may be bindings between a single residue and separate ligand fragments. As an example, the bindings 910c, 910d, and 910e may be expressed as a plurality of binding words, but may be expressed as the single binding sentence 920c generated by combining the plurality of binding words.

As another example, in Val-726, 910f and 910g may also be considered as bindings formed by the single binding atom within the single protein residue with different binding atoms within the single ligand. Through this, similar conclusions to 910c, 910d, and 910e may also be derived for the bindings 910f and 910g in terms of expression as the binding word and/or the binding sentence. The bindings 910f and 910g may be bindings between the single residue and separate ligand fragments. As an example, the bindings 910f and 910g may be expressed as a plurality of binding words, but may be expressed as the single binding sentence 920c generated by combining the plurality of binding words.

As additional examples, it is considered that the bindings 910c, 910d, and 910e and the bindings 910f and 910g are compared from a perspective of the binding word and/or the binding sentence. As an example, receptor binding atoms of the bindings 910c, 910d, and 910e may be different from the receptor binding atoms of the bindings 910f and 910g. However, the different receptor binding atoms are considered to be part of the single protein residue, Val-726. Therefore, according to the definition of the binding sentence in the present disclosure, 910c, 910d, 910e, 910f, and 910g may express the single sentence 920c by a combination of binding words expressing the respective bindings.

In an embodiment of the present disclosure, the binding paragraph may express a binding structure between a single protein binding pocket and each of all ligand fragments that are bound to the binding pocket. As an example, the binding paragraph may be generated by combining a plurality of binding sentences. As an example, the structures 910a to 910j of all bindings included in the protein-ligand complex of FIG. 9 may be expressed as the single binding paragraph 920. As an example, the binding paragraph 920 may be a combination of the plurality of binding sentences 920a, 920b, and 920c.

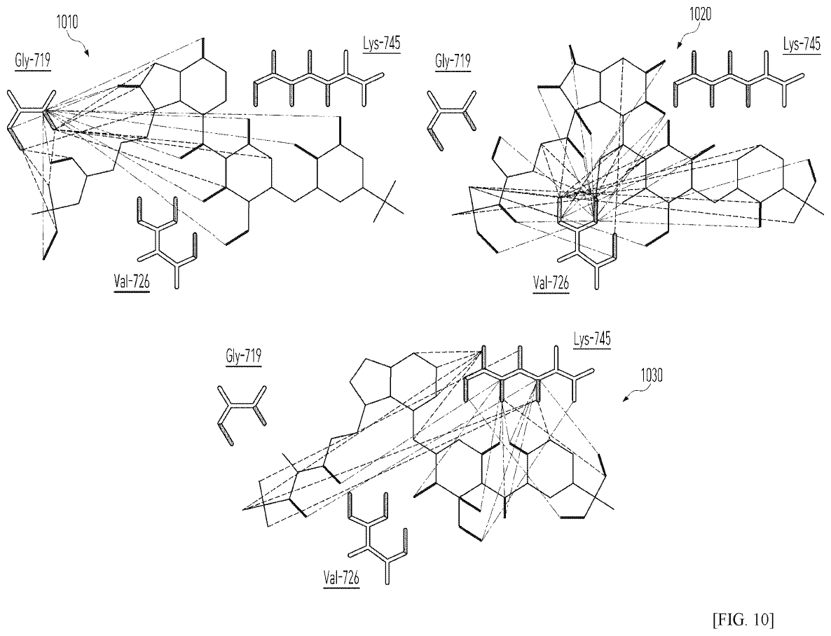

FIG. 10 illustrates an example of bindings constituting binding sentences according to an embodiment of the present disclosure. The bindings illustrated in FIG. 10 may be considered as additional bindings included in the protein-ligand complex 910 of FIG. 9. As an example, the binding between Gly-719 and the ligand may include bindings depicted by reference numeral 1010. The bindings included in reference numeral 1010 may form a plurality of binding words, and a single binding sentence may be expressed as an extended form of the binding sentence 920a illustrated in FIG. 9.

As an example, the binding between Val-726 and the ligand may include bindings depicted by reference numeral 1020. The bindings included in reference numeral 1020 may form a plurality of binding words, and the single binding sentence may be expressed as an extended form of the binding sentence 920c illustrated in FIG. 9.

As an example, the binding between Lys-745 and the ligand may include bindings depicted by reference numeral 1030. The bindings included in reference numeral 1030 may form a plurality of binding words, and the single binding sentence may be expressed as an extended form of the binding sentence 920b illustrated in FIG. 9.

FIG. 11 exemplarily illustrates a clustering result of an embedding vector extracted from a trained large language model according to an embodiment of the present disclosure.

According to an embodiment of the present disclosure, the computing device 100 may generate training data including the binding words, the binding sentences, and/or the binding paragraphs described above. The computing device 100 may train a large language model using training data. The computing device 100 may obtain an embedding vector corresponding to an input query using the trained large language model.

According to an embodiment of the present disclosure, when generating binding sentences in the trained large language model, the computing device 100 may generate an embedding vector representing each of the binding sentences. As an example, when the trained large language model generates a binding sentence, the trained large language model may generate tokens constituting the binding sentence. As an example, the trained large language model may generate a probability distribution embedding vector corresponding to each of the tokens. As an example, the probability distribution embedding vector may be extracted from the last hidden layer of the model. As an example, the computing device 100 may calculate an average of probability distribution embedding vectors for all tokens constituting the binding sentence. As an example, the calculated average value of the embedding vectors may be determined as a representative embedding vector of the generated binding sentence.

According to an embodiment of the present disclosure, a binding word may be defined, which represents a key binding within a specific binding sentence.

As an example, when the trained large language model generates a binding sentence, the trained large language model may generate embedding vectors corresponding to the tokens constituting the binding sentence. As an example, the embedding vector may be extracted from the last hidden layer of the model.

As an example, an Average of Vectors (AV) of embedding vectors corresponding to all tokens constituting the binding sentence may be obtained.

As an example, a Frequency of Word (FW) may be calculated for each of the binding words that constitute the binding sentence. The frequency of word may be defined as a value obtained by dividing the number of specific binding words included in a plurality of binding sentences by the number of plurality of binding sentences. As an example, an FW filter may be applied in such a scheme of excluding a binding word with FW<0.05 from the key binding.

As an example, among binding words that are not excluded by the FW filter, a binding word that satisfies a condition AV>0.5 may be defined as a binding word indicating the key binding.

According to an embodiment of the present disclosure, the computing device 100 may perform an operation of extracting a plurality of first embedding vectors from at least one layer of the trained LLM.

As an example, a layer of a large language model is a function that converts or processes input data into a specific form, and is composed of parameters such as weights and biases, allowing the large language model to efficiently learn input data and generate an output. As an example, the layer may include an input layer that converts or preprocesses a form of the input data. As an example, the layer may include an output layer that generates a final output of the model and performs prediction or classification of the model for a given input. As an example, the layer may include a hidden layer, which is an intermediate layer between the input layer and the output layer and is used to extract features of data or learn complex patterns.

As an example, the computing device 100 may extract an embedding vector from the hidden layer. As an example, the computing device 100 may extract the embedding vector from the last hidden layer.

As an example, in the large language model, embedding may be a process of converting text data into a high-dimensional vector space. Through the embedding, a unique vector is assigned to each word and/or token to represent a meaning and characteristics of the word. As an example, the embedding vector may be a vector for each word and/or token generated via such embedding. As an example, the embedding vector may be constituted by high-dimensional real number value, and each dimension may represent a specific characteristic or meaning of the word. As an example, a first embedding vector may be a high-dimensional vector generated through the embedding.

According to an embodiment of the present disclosure, the computing device 100 may perform an operation of obtaining a plurality of second embedding vectors by reducing dimensions of respective first embedding vectors. As an example, the dimensionality reduction may be converting the high-dimensional vector generated through the embedding into a low-dimensional vector. As an example, principal component analysis (PCA), t-SNE, and/or UMAP may be used as techniques for the dimensionality reduction. As an example, the second embedding vector may be a vector converted into a low-dimensional value through the dimensionality reduction technique.

According to an embodiment of the present disclosure, the computing device 100 may perform clustering of second embedding vectors or calculating a distance between each of the second embedding vectors in a vector space. As an example, clustering may be a process of grouping vectors that have similar characteristics. As an example, by calculating the distance between vectors, vectors with a distance equal to or less than a threshold value may be determined as the vectors with similar characteristics. As an example, a method for calculating the distance between vectors may include an Euclidean distance. Here, the Euclidean distance may be a method for measuring a straight distance between two points by utilizing the Pythagorean theorem. As an example, the method for calculating the distance between vectors may include a cosine similarity. Here, the cosine similarity which is a method for determining how similar the two vectors are facing by measuring an angle between two vectors may be calculated as a value obtained by dividing inner products of the two vectors by a product of magnitudes of the vectors.

In an embodiment, a distance-based clustering algorithm may be used to cluster the second embedding vectors. As an example, the distance-based clustering algorithm may include K-means clustering, Density-Based Clustering, Density-Based Spatial Clustering of Applications with Noise (DBSCAN), and/or Hierarchical Clustering.

In an embodiment, a graph-based clustering algorithm may be used to cluster the second embedding vectors. As an example, the graph-based clustering algorithm may include Louvain clustering.

FIG. 11 illustrates a result of clustering an embedding vector with a reduced dimension according to an embodiment of the present disclosure. A graph 1110 illustrates a result of reducing a dimension of an embedding vector corresponding to a plurality of binding data by using UMAP, and then clustering the embedding vector with reduced dimension by a Louvain clustering algorithm according to an embodiment of the present disclosure. The graph 1110 illustrates a result of grouping the embedding vectors corresponding to the binding data into 23 clusters through the clustering. By referring to legend 1120, each cluster in each graph may be distinguished. The legend 1120 may express a number or identification information for each cluster. One point in FIG. 11 may express an embedding vector corresponding to at least one query.

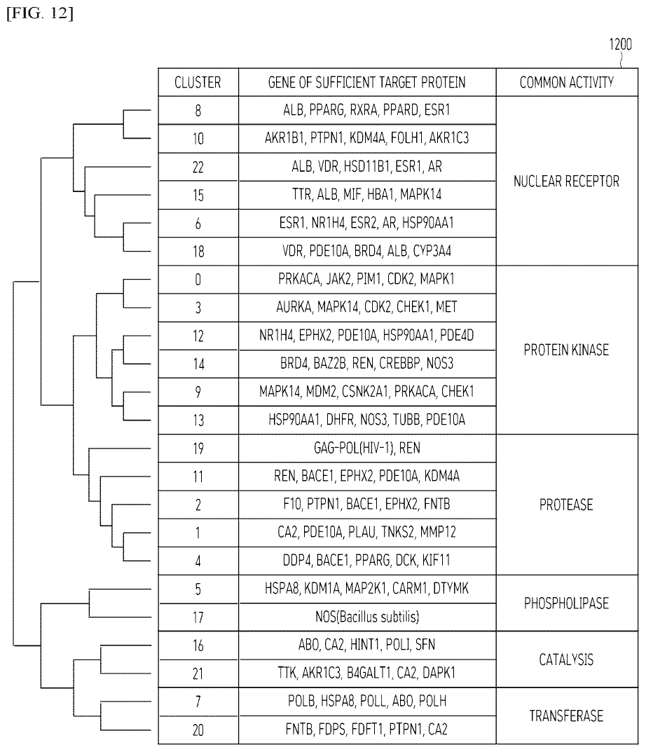

FIG. 12 exemplarily illustrates a result of layering clustering results and sorting the layered clustering results for each biological function according to an embodiment of the present disclosure. As an example, the layering may be a result of the hierarchical clustering. As an example, hierarchical clustering may operate by a scheme of forming a high-level cluster by grouping clusters based on a distance or a similarity between data points.

As an example, a cluster number in column 1 of table 1200 of FIG. 12 corresponds to a cluster number or cluster identification information 1120 illustrated in FIG. 11. As an example, the cluster numbers may be rearranged in an order in which the cluster numbers are grouped into the same upper cluster through hierarchical clustering. As an example, a result of the hierarchical clustering is illustrated on a left side of the table 1200.

As an example, column 2 of table 1200 of FIG. 12 shows gene types of proteins corresponding to the embedding vectors belonging to each cluster. As an example, the genes in column 2 above may be listed in order of their frequency of existence within each cluster.

As an example, each of the clustered embedding vectors may include annotated data. As an example, the annotated data may include identification information for each protein that forms the binding structure. As an example, the identification information of the protein may be assigned through UniProt ID mapping. As an example, through the UniProt ID mapping, the identification information of the protein may be converted into identification information of a gene encoding the protein.

In an embodiment, a function or activity of the protein may be determined by reference to the identification information of the gene. Column 3 of table 1200 in FIG. 12 shows a common function of the genes listed in column 2. The common function in column 3 may tend to match the hierarchical clustering result on the left side of column 1.

In an embodiment of the present disclosure, the technique utilizing the clusters obtained using the artificial intelligence model as such may be useful for determining a common function of a plurality of proteins.

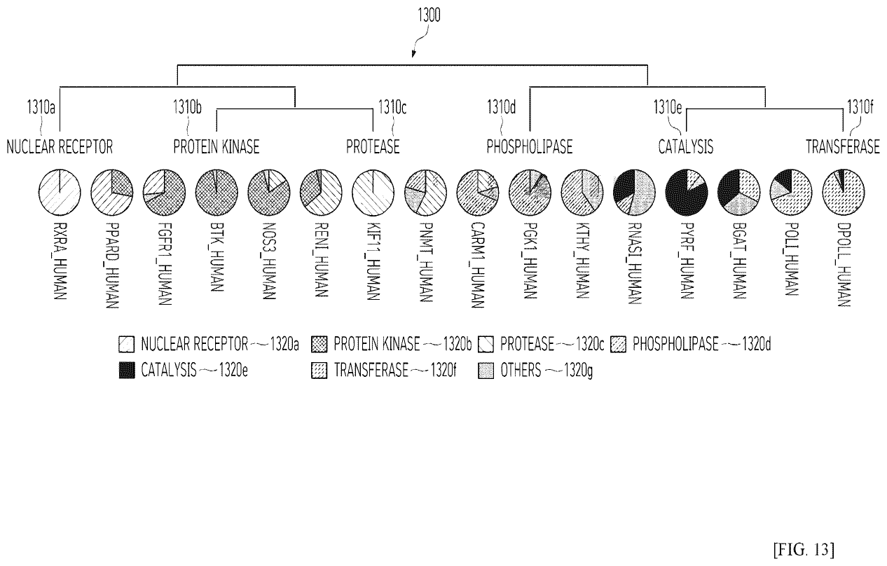

FIG. 13 exemplarily illustrates a result in which proteins classified as multifunctional proteins are scattered in multiple clusters according to an embodiment of the present disclosure.

In an embodiment, embedding vectors that share identification information of a single protein may be included in the same cluster through the clustering. In an embodiment, in contrast, the embedding vectors that share the identification information of the single protein may be scattered in a plurality of clusters.

FIG. 13 illustrates a pie graph of some proteins according to a hierarchical clustering result. Through the pie graph, it is possible to confirm which upper cluster among the upper clusters represented by the common function illustrated through column 3 of FIG. 12 (1200) the embedding vectors corresponding to each protein belong to. As an example, an RXRA_HUMAN protein and a KIF11_HUMAN protein may be considered to have most of their points included in a nuclear receptor (1310a) group and a protease (1310c) group, respectively. In contrast, a PPARD_HUMAN protein may be considered to have points across an upper cluster of a nuclear receptor 1320a and protein kinase (1320b), and an RENI_HUMAN protein may be considered to have points across an upper cluster of the protein kinase 1320b and protease (1320c). The method including the examples through hierarchical clustering may provide an advanced perspective in analyzing the similarity of characteristics of a plurality of proteins. As an example, the similarity of the characteristics may include structural characteristics of the protein, functional characteristics of the protein, and genetic characteristics of the protein.

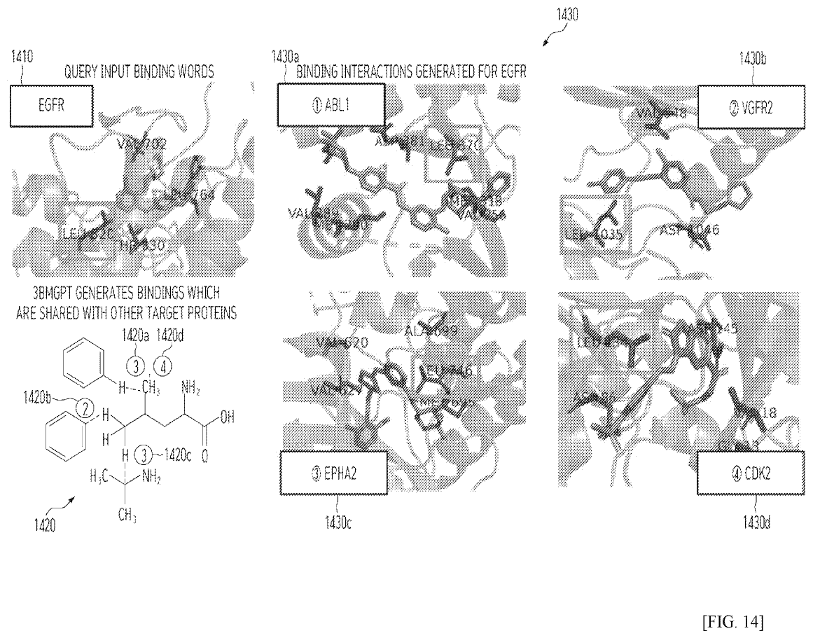

FIG. 14 illustrates an example in which shared bindings with other proteins are output when a query is input into a large language model trained by an embodiment of the present disclosure.

In an embodiment, an algorithm which returns a three-dimensional structure corresponding to a specific query may additionally be implemented. In an embodiment, the specific query may be a plurality of binding words. In an embodiment, the specific query may be a specific binding sentence.

In an embodiment, a three-dimensional structure of identification information corresponding to pre-trained text-form data may be paired with the large language model. In an embodiment, the paired information may be databased.

In an embodiment, the three-dimensional structure of identification information may include genetic identification information of the protein. In an embodiment, the three-dimensional structure of identification information may include identification information of the protein-ligand complex.

In an embodiment, binding data converted from binding poses of the protein-ligand complex may be stored as a sub-data structure in the three-dimensional structure of identification information. As an example, the converted binding data may include a plurality of binding words. As an example, the converted binding data may be a combination of characteristic binding words corresponding to the three-dimensional structure.

In an embodiment, an algorithm may be defined to primarily filter identification information of three-dimensional structure that are most including binding words included in query input data as sub-data. In an embodiment, an algorithm may be additionally defined to select, among the filtered identification structures, a structure having a high exact match count between the sub-data and the query as a representative three-dimensional structure.

In an embodiment, the query may be input into the trained large language model. In an embodiment, the query may be a plurality of binding words representing a binding structure of EGFR, a kinase, and 5N3, a ligand that is bound to EGFR. Reference numeral 1410 may be a 3D modeling depicting bindings corresponding to the binding words of the EGFR-5N3 as the query input.

In an embodiment, the trained large language model may generate separate binding words as an output for the query (1420). In an embodiment, the binding words as the output may be binding words corresponding to proteins other than EGFR. In an embodiment, the binding words as the output may represent separate proteins that share a binding structure similar to that of the binding of the input EGFR-SN3 (1430). In an embodiment, the binding words as the output may include binding words corresponding to ABL1 (1420a, 1430a), VGFR2 (1420b, 1430b), EPHA2 (1420c, 1430c), and/or CDK2 (1420d, 1430d). Reference numerals 1430a, 1430b, 1430c, and 1430d may be results of 3D modeling the binding structure of the binding words returned as the output. In an embodiment, ABL1, VGFR2, EPHA2, and/or CDK2 may be considered proteins that share similar bindings to EGFR.

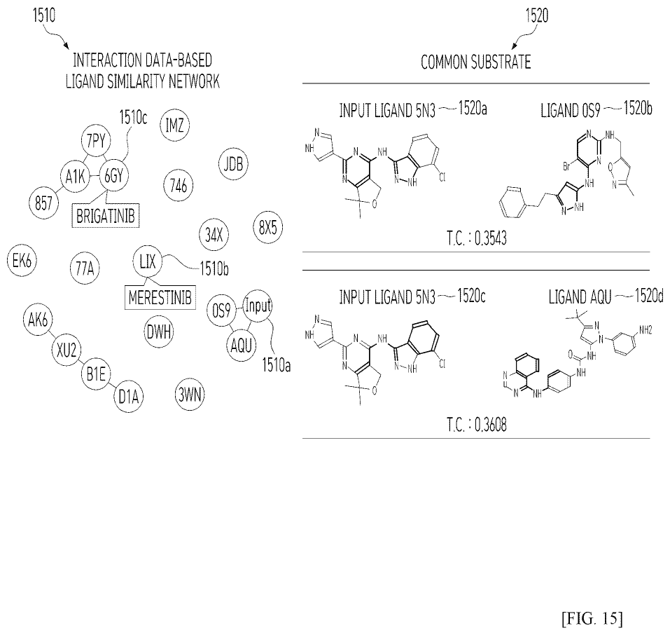

FIG. 15 exemplarily illustrates a result of determining a ligand that may be used as a common substrate from the results returned by the large language model trained by an embodiment of the present disclosure.

In an embodiment, an analysis for binding ligands may be additionally performed from the example of FIG. 14 in which the binding words corresponding to EGFR-SN3 are input. In an embodiment, the annotated training data may include ligand identification information via PDB ID mapping.

In an embodiment, to analyze the molecular characteristics of a ligand 5N3, which is input as the query in the example of FIG. 14, 20 data points having a close distance to an input query 1510a in the vector space are illustrated (1510). The 20 data points illustrated may be considered as ligands having a similarity.

As an example, ligand AQU 1520d and ligand 0S9 1520b may be considered as ligands having a closest distance to the input ligand 5N3 (1520a and 1520c). As an example, a Tanimoto coefficient may be calculated to validate similarities between the AQU and 0S9, and 5N3 (1520).

As an example, among the 20 data illustrated, ligand 6GY with the pharmaceutical name brigatinib 1510c and ligand L1X with the pharmaceutical name merestinib 1510b are included (1510). Brigatinib is used as a drug that inhibits anaplastic lymphoma kinase. As an example, merestinib is used as a drug that inhibits neurotrophic receptor kinase. Both brigatinib and merestinib are considered as cancer treatments. As an example, target proteins of brigatinib and merestinib are both associated with tyrosine kinase, and the protein EGFR corresponding to the input binding word is also classified as tyrosine kinase. As described above, the method according to the present disclosure may be used to analyze the similarity of characteristics of ligands. As an example, the similarity of the characteristics of the ligands may include a similarity in structural characteristics and a similarity in functional characteristics. According to an embodiment of the present disclosure, the analysis of the similarity may be used to determine an additional use of the drug.

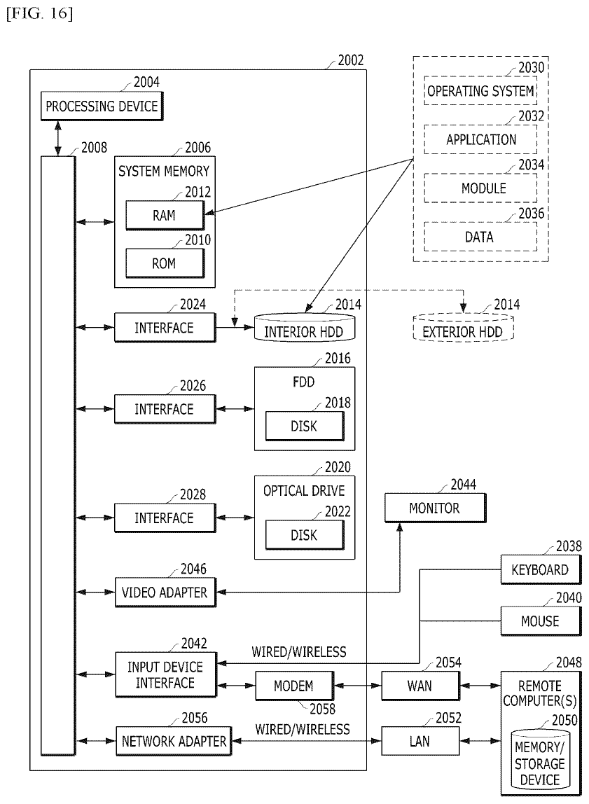

FIG. 16 is a schematic view of a computing environment according to an embodiment of the present disclosure.

In general, the component, module or unit in the present specification includes a routine, a procedure, a program, a component, a data structure, and the like that execute a specific task or implement a specific abstract data type. Further, those skilled in the art will appreciate well that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer.

The exemplary embodiments of the present disclosure may be carried out in a distribution computing environment, in which certain tasks are performed by remote processing devices connected through a communication network. In the distribution computing environment, a program module may be located in both a local memory storage device and a remote memory storage device.

The computing device generally includes various computer readable media. A computer accessible medium may be a computer readable medium regardless of the kind of medium. The computer readable medium includes volatile and non-volatile media and transitory and non-transitory media, and portable and non-portable media. As a non-limited example, the computer readable medium may include a computer readable storage medium and a computer readable transport medium.

The computer readable storage medium includes volatile and non-volatile media, transitory and non-non-transitory media, and portable and non-portable media constructed by any method or technology, which stores information, such as a computer readable command, a data structure, a program module, or other data. The computer storage medium includes a read only memory (RAM), a read only memory (ROM), electrically erasable and programmable ROM (EEPROM), a flash memory, or other memory technologies, a compact disc (CD)-ROM, a digital video disk (DVD), or other optical disk storage devices, a magnetic cassette, a magnetic tape, a magnetic disk storage device, or other magnetic storage device, or any other media, which are accessible by a computer and are used for storing desired information, but is not limited thereto.

The computer readable transport medium generally includes all of the information transport media, such as other transport mechanisms, which implement a computer readable command, a data structure, a program module, or other data in a modulated data signal. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, radio frequency (RF), infrared rays, and other wireless media. A combination of any media among the foregoing media is also included in a range of the computer readable transport medium.

An illustrative environment (2000) including a computer (2002) and implementing several aspects of the present disclosure is illustrated, and the computer (2002) includes a processing device (2004), a system memory (2006) and a system bus (2008) The computer (200) in this disclosure may be exchangeable with computing device. The system bus (2008) connects system components including the system memory (2006) (not limited thereto) to the processing device (2004). The processing device (2004) may be any processor among various common processors. A dual processor and other multi-processor architectures may also be used as the processing device (2004).

The system bus (2008) may be a predetermined one among several types of bus structure, which may be additionally connectable to a local bus using a predetermined one among a memory bus, a peripheral device bus, and various common bus architectures. The system memory (2006) includes a ROM (2010), and a RAM (2012). A basic input/output system (BIOS) is stored in a non-volatile memory (2010), such as a ROM, an erasable and programmable ROM (EPROM), and an EEPROM, and the BIOS includes a basic routine helping a transport of information among the constituent elements within the computer (2002) at a time, such as starting. The RAM (2012) may also include a high-rate RAM, such as a static RAM, for caching data.

The computer (2002) also includes an embedded hard disk drive (HDD) (2014) (for example, enhanced integrated drive electronics (EIDE) and serial advanced technology attachment (SATA)), a magnetic floppy disk drive (FDD) (2016) (for example, which is for reading data from a portable diskette (2018) or recording data in the portable diskette 2018), SSD and an optical disk drive (2020) (for example, which is for reading a CD-ROM disk (2022), or reading data from other high-capacity optical media, such as a DVD, or recording data in the high-capacity optical media). A hard disk drive (2014), a magnetic disk drive (2016), and an optical disk drive (2020) may be connected to a system bus (2008) by a hard disk drive interface (2024), a magnetic disk drive interface (2026) and an optical drive interface (2028), respectively. An interface (2024) or implementing an outer mounted drive includes at least one of or both a universal serial bus (USB) and the Institute of Electrical and Electronics Engineers (IEEE) 1394 interface technology.

The drives and the computer readable media associated with the drives provide non-volatile storage of data, data structures, computer executable commands, and the like. In the case of the computer (2002), the drive and the medium correspond to the storage of predetermined data in an appropriate digital form. In the description of the computer readable storage media, the HDD, the portable magnetic disk, and the portable optical media, such as a CD, or a DVD, are mentioned, but those skilled in the art will appreciate well that other types of computer readable storage media, such as a zip drive, a magnetic cassette, a flash memory card, and a cartridge, may also be used in the illustrative operation environment, and the predetermined medium may include computer executable commands for performing the methods of the present disclosure.

A plurality of program modules including an operation system (2030), one or more application programs (2032), other program modules (2034), and program data (2036) may be stored in the drive and the RAM (2012). An entirety or a part of the operation system, the application, the module, and/or data may also be cached in the RAM (2012). It will be appreciated that the present disclosure may be implemented by several commercially usable operation systems or a combination of operation systems.

A user may input a command and information to the computer (2002) through one or more wired/wireless input devices, for example, a keyboard (2038) and a pointing device, such as a mouse (2040). Other input devices (not illustrated) may include a microphone, an IR remote controller, a joystick, a game pad, a stylus pen, a touch screen, and the like. The foregoing and other input devices are frequently connected to the processing device (2004) through an input device interface (2042) connected to the system bus (2008), but may be connected by other interfaces, such as a parallel port, an IEEE 1394 serial port, a game port, a USB port, an IR interface, and other interfaces.

A monitor (2044) or other types of display devices are also connected to the system bus (2008) through an interface, such as a video adapter (2046). In addition to the monitor (2044), the computer generally includes other peripheral output devices (not illustrated), such as a speaker and a printer.

The computer (2002) may be operated in a networked environment by using a logical connection to one or more remote computers, such as remote computer(s) 2048, through wired and/or wireless communication. The remote computer(s) 2048 may be a workstation, a computing device computer, a router, a personal computer, a portable computer, a microprocessor-based entertainment device, a peer device, and other general network nodes, and generally includes some or an entirety of the constituent elements described for the computer 2002, but only a memory storage device 2050 is illustrated for simplicity. The illustrated logical connection includes a wired/wireless connection to a local area network (LAN) 2052 and/or a larger network, for example, a wide area network (WAN) 2054. The LAN and WAN networking environments are general in an office and a company, and make an enterprise-wide computer network, such as an Intranet, easy, and all of the LAN and WAN networking environments may be connected to a worldwide computer network, for example, Internet.

When the computer 2002 is used in the LAN networking environment, the computer 2002 is connected to the local network 2052 through a wired and/or wireless communication network interface or an adapter 2056. The adapter 2056 may make wired or wireless communication to the LAN 2052 easy, and the LAN 2052 may also include a wireless access point installed therein for the communication with the wireless adapter 2056. When the computer 2002 is used in the WAN networking environment, the computer 2002 may include a modem 2058, or includes other means connected to a communication computing device in the WAN 2054 or setting communication through the WAN 2054 via the Internet and the like. The modem 2058, which may be an embedded or outer-mounted and wired or wireless device, is connected to the system bus 2008 through a serial port interface 2042. In the networked environment, the program modules described for the computer 2002 or some of the program modules may be stored in a remote memory/storage device 2050. The illustrated network connection is illustrative, and those skilled in the art will appreciate well that other means setting a communication link between the computers may be used.

The computer 2002 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes a wireless fidelity (Wi-Fi) and Bluetooth wireless technology at least. Accordingly, the communication may have a pre-defined structure, such as a network in the related art, or may be simply ad hoc communication between at least two devices.

It shall be understood that a specific order or a hierarchical structure of the operations included in the presented processes is an example of illustrative accesses. It shall be understood that a specific order or a hierarchical structure of the operations included in the processes may be re-arranged within the scope of the present disclosure based on design priorities. The accompanying method claims provide various operations of elements in a sample order, but it does not mean that the claims are limited to the presented specific order or hierarchical structure.

### Mode for Invention

Related contents in the best mode for carrying out the present disclosure are described as above.

### Industrial Applicability

The present disclosure may provide an artificial intelligence model for analyzing and/or predicting an interaction between a protein and a ligand by preprocessing a three-dimensional binding structure between the protein and the ligand to be processed by the artificial intelligence model.

1. A method performed by a computing device, comprising;
converting, a binding structure between a ligand and a protein into at least one binding word in text form which is processable in an artificial intelligence-based Large Language Model;
generating, training data using the at least one binding word; and
training, the Large Language Model using the training data.

2. The method of claim 1, wherein the converting comprises,
converting the binding structure between a binding part of the ligand and a residue of the protein into the at least one binding word.

3. The method of claim 2, wherein the converting comprises,
converting the binding structure, which represents an interaction of an electron donor and an electron acceptor between the binding part of the ligand and the residue of the protein into, the at least one binding word.

4. The method of claim 2, wherein the binding word comprises:
a first sub-binding word representing an interaction from a perspective of the ligand on the binding structure; and
a second sub-binding word representing an interaction from a perspective of the protein on the binding structure.

5. The method of claim 4, wherein the converting comprises:
converting the binding structure between the ligand and the protein into the at least one binding word by concatenating the first sub-binding word and the second sub-binding word.

6. The method of claim 4, wherein the first sub-binding word comprises:
a first part representing whether a role of a binding atom of the ligand on the binding structure is an electron donor or an electron acceptor;
a second part identifying the binding atom of the ligand on the binding structure; and
a third part identifying at least one proximal atom located proximal to the binding atom of the ligand on the binding structure.

7. The method of claim 4, wherein the first sub-binding word is a concatenated form of:
a first information representing whether a role of a binding atom of the ligand on the binding structure is an electron donor or an electron acceptor;
a second information identifying a binding form formed by the binding atom of the ligand with other atoms of the ligand on the binding structure;
a third information identifying a first proximal atom located proximal to the binding atom of the ligand and a binding form formed by the first proximal atom with other atoms of the ligand on the binding structure;
a fourth information identifying the binding atom of the ligand and the binding form formed by the binding atom with other atoms of the ligand on the binding structure; and
a fifth information identifying a second proximal atom located proximal to the binding atom of the ligand and a binding form formed by the second proximal atom with other atoms of the ligand on the binding structure.

8. The method of claim 4, wherein the second sub-binding word comprises:
a first part identifying a binding amino acid of the protein on the binding structure;
a second part identifying a receptor binding atom obtained from the binding amino acid; and
a third part identifying at least one receptor proximal atom located proximal to the receptor binding atom.

9. The method of claim 8, wherein the at least one receptor proximal atom comprises a first receptor proximal atom and a second receptor proximal atom, and
in the third part identifying the at least one receptor proximal atom located proximal to the receptor binding atom, the first receptor proximal atom, the receptor binding atom, and the second receptor proximal atom are concatenated in the order of the first receptor proximal atom, the receptor binding atom, and the second receptor proximal atom.

10. The method of claim 4, wherein the first sub-binding word and the second sub-binding word, which constitute the binding word, are concatenated through a first expression to represent the binding word;
the parts or information which constitute the first sub-binding word are concatenated through a second expression to represent the first sub-binding word;
the parts or information which constitute the second sub-binding word are concatenated through the second expression to represent the second sub-binding word; and
the first expression and the second expression are different from each other.

11. The method of claim 1, wherein the binding structure between the ligand and the protein is a three-dimensional binding structure, and the at least one binding word is one-dimensional data.

12. The method of claim 1, wherein the generating the training data comprises:
generating a binding sentence representing a binding structure between a single protein residue and all ligand fragments binding to the single protein residue by combining a plurality of binding words; and
generating the training data including the binding sentence.

13. The method of claim 12, wherein the generating the training data comprises:
generating a binding paragraph representing a binding structure between a binding pocket of a single protein and each ligand fragment binding to the binding pocket by combining a plurality of binding sentences; and
generating the training data including the binding paragraph.

14. The method of claim 12, wherein the generating the training data including the binding sentence comprises:
generating the training data by annotating identification information of protein, species information, and identification information of ligand corresponding to each of the binding sentences.

15. The method of claim 13, wherein the training the Large Language Model using the training data comprises tokenizing the training data, and
wherein the tokenizing comprises:
a word-based tokenization, in which each of the binding words acts as a token to form a vocabulary; and
a byte-pair encoding tokenization, in which all words included in each binding paragraph are connected by a space character, and each binding paragraph is separated by a newline character.

16. The method of claim 1, further comprising:
extracting a plurality of first embedding vectors from at least one layer of the trained Large Language Model;
reducing dimensionality of each of the first embedding vectors to obtain a plurality of second embedding vectors;
clustering the second embedding vectors or calculating a distance of each of the second embedding vectors in vector space; and
determining a similarity of characteristics of proteins, a similarity of characteristics of ligands, and a binding potential between a protein and a ligand based on a result of the clustering or the distance, and
wherein the characteristics comprise structural characteristics, functional characteristics, and genetic characteristics.

17. The method of claim 16, further comprising:
determining a single protein as a multi-functional protein when a plurality of embedding vectors corresponding to the single protein exist in a plurality of clusters as a result of the clustering.

18. The method of claim 16, further comprising:
determining additional uses of drugs corresponding to the ligands using the similarity of the characteristics of ligands.

19. A computer program stored in a non-transitory computer readable storage medium, wherein the computer program causes a computing device to perform following operations when executed by the computing device, wherein the operation comprises:
converting, a binding structure between a ligand and a protein into at least one binding word in text form which is processable in an artificial intelligence-based Large Language Model;
generating, training data using the at least one binding word; and
training, the Large Language Model using the training data.

20. A computing device comprising:
at least one processor; and
a memory; and
wherein the at least one processor performs:
converting, a binding structure between a ligand and a protein into at least one binding word in text form which is processable in an artificial intelligence-based Large Language Model;
generating, training data using the at least one binding word; and
training, the Large Language Model using the training data.
